(19) United States
(12) Patent Application Publication
(10) Pub. No.: US 2026/0053407 A1
(43) Pub. Date: Feb. 26, 2026

(54) DRIVING STATE MONITORING AND FEEDBACK METHOD AND SYSTEM BASED ON MULTIMODAL HUMAN-FACTORS INTELLIGENT DATA ANALYSIS, AND EDGE COMPUTING TERMINAL DEVICE

(71) Applicant: KINGFAR INTERNATIONAL INC., Beijing (CN)

(72) Inventors: Qichao ZHAO, Beijing (CN); Qingju WANG, Beijing (CN); Ran YANG, Beijing (CN)

(21) Appl. No.: 19/373,784

(22) Filed: Oct. 30, 2025

Related U.S. Application Data
(63) Continuation of application No. PCT/CN2024/131835, filed on Nov. 13, 2024.

(30) Foreign Application Priority Data
Dec. 5, 2023 (CN) 202311659868.8
Dec. 21, 2023 (CN) 202311775914.0
Dec. 22, 2023 (CN) 202311782765.0
Dec. 29, 2023 (CN) 202311865457.4

(51) Int. Cl. A61B 5/18; A61B 5/00; B60Q 9/00; G06V 10/30; G06V 10/764; G06V 10/98; G06V 20/40; G06V 20/56; G06V 20/59; G06V 40/16; G06V 40/18; G06V 40/20; G07C 5/04

(52) U.S. Cl. CPC A61B 5/18; A61B 5/0077; A61B 5/7203; B60Q 9/00; G06V 10/30; G06V 10/764; G06V 10/993; G06V 20/41; G06V 20/588; G06V 20/597; G06V 40/171; G06V 40/174; G06V 40/193; G06V 40/20; G07C 5/04; A61B 2576/00

(57) ABSTRACT

Provided are a driving state monitoring and feedback method and system based on multi-modal human-factor intelligent data analysis, and an edge computing terminal device. The method includes: receiving multi-modal human-factor data collected in real time from a tested driver; preprocessing the multi-modal human-factor data; inputting the preprocessed multi-modal human-factor data to a pre-trained first state identification model to obtain a driver state identified in real-time, the driver state including a normal state and a plurality of abnormal states; and generating, in response to the driver state being identified as an abnormal state, a driving state feedback instruction for the category of the abnormal state and sending the driving state feedback instruction to a driving intervention system, to cause the driving intervention system to perform state feedback adjustment on the driver based on the received driving state feedback instruction.

### CROSS-REFERENCE TO RELATED APPLICATION

This application is a continuation of International Patent Application No. PCT/CN2024/131835, filed on Nov. 13, 2024, which based on and claims priorities to Chinese Patent Application No. 202311775914.0 filed on Dec. 21, 2023, Chinese Patent Application No. 202311782765.0 filed on Dec. 22, 2023, Chinese Patent Application No. 202311865457.4 filed on Dec. 29, 2023, and Chinese Patent Application No. 202311659868.8 filed on Dec. 5, 2023, which are incorporated herein by reference in their entireties.

### FIELD

The present disclosure relates to the field of driving technologies, and more particularly, to a driving state monitoring and feedback method and system based on multi-modal human-factor intelligent data analysis, and an edge computing terminal device.

### BACKGROUND

With the rapid growth of road traffic demand, traffic safety issues are becoming increasingly prominent. As the main controller, regulator, and information decision-maker of a road traffic system, the driver has always been the focus of research on road traffic safety. The monitoring and feedback of the driving state of a vehicle driver (e.g., identifying the presence of issues such as fatigued driving, distracted driving, and angry driving) is a key research direction in the field of road traffic safety driving intervention, while how to perform monitoring and feedback on the driving state of the vehicle driver is an urgent technical problem to be solved.

### SUMMARY

Embodiments of the present disclosure provides a driving state monitoring and feedback method and system based on multi-modal human-factors intelligent data analysis, and an edge computing terminal device.

In an aspect of the present disclosure, a driving state monitoring and feedback method based on multi-modal human-factor intelligent data analysis is provided. The method includes: receiving multi-modal human-factor data collected in real time from a tested driver; preprocessing the multi-modal human-factor data, the preprocessing including noise reduction processing and data normalization processing; inputting the preprocessed multi-modal human-factor data to a pre-trained first state identification model to obtain a driver state identified in real-time, the driver state including a normal state and a plurality of abnormal states, a category of the plurality of abnormal states including one or more of a fatigue state, a distracted state, and an angry state; and generating, in response to the driver state being identified as an abnormal state among the plurality of abnormal states, a driving state feedback instruction for the category of the abnormal state and sending the driving state feedback instruction to a driving intervention system, to cause the driving intervention system to perform state feedback adjustment on the driver based on received driving state feedback instruction.

In another aspect of the present disclosure, a driving state monitoring and feedback system based on multi-modal human-factor intelligent data analysis is provided. The system includes: a state identification subsystem configured to receive multi-modal human-factor data collected in real time from a tested driver, performs preprocessing on the multi-modal human-factor data; and input the preprocessed multi-modal human-factor data to a pre-trained first state identification model to obtain a driver state identified in real-time, the human-factor data including at least two of electroencephalographic data, heart rate data, electrodermal activity data, respiratory data, near-infrared data, blood oxygen data, blood pressure data, and skin temperature data, the preprocessing including noise reduction processing and data normalization processing, the driver state including a normal state and a plurality of abnormal states, and a category of the plurality of abnormal states including at least two of a fatigue state, a distracted state, and an angry state; and a driving intervention subsystem configured to generate, in response to the driver state being identified as an abnormal state, a driving state feedback instruction for the category of the abnormal state and send the driving state feedback instruction to a driving intervention system, to cause the driving intervention system to perform state feedback adjustment on the driver based on received driving state feedback instruction. The first state identification model is obtained by collecting baseline human-factor data and demographic data of the tested driver, retrieving, from a baseline state database, driver-related data having a similarity with the baseline human-factor data and the demographic data of the tested driver that is within a predetermined similarity range, and performing iterative training by using retrieved baseline human-factor data of the driver and driver state data stored in the baseline state database as a training set and using a pre-determined normal state and a plurality of pre-determined abnormal states as labels.

In another aspect of the present disclosure, an edge computing terminal device is provided. The edge computing terminal device includes a processor; and a memory having computer instructions stored thereon. The processor is configured to execute the computer instructions stored in the memory. The device is configured to, when the computer instructions are executed by the processor, implement the method according to any one of the above-described embodiments.

In another aspect of the present disclosure, a computer-readable storage medium is provided. The computer-readable storage medium has a computer program stored thereon. The computer program, when executed by a processor, implements the method according to any one of the above-described embodiments.

The driving state monitoring and feedback method and system based on multi-modal human-factor intelligent data analysis and the edge computing terminal device provided in the present disclosure can process the pre-processed multi-modal human-factor data collected in real time by using the pre-trained first state identification model, distinguish the normal state and abnormal state of the driver through classification labels, identify different driving states of the driver, and process the different driving states to avoid traffic accidents.

Additional advantages, objects, and features of the present disclosure will be explained at least in part in the following description, and will become apparent to those skilled in the art upon examination of the following description, or can be learned from practicing of the present disclosure. The objects and other advantages of the present disclosure can be achieved and obtained by means of structures specifically pointed out in the specification and the accompanying drawings.

It will be appreciated by those skilled in the art that the objects and the advantages that can be achieved by the present disclosure are not limited to the above specific description. The above and other objects that can be achieved by the present disclosure will be more clearly understood from the following detailed description.

### BRIEF DESCRIPTION OF THE DRAWINGS

In order to clearly explain technical solutions of embodiments of the present disclosure, drawings used in the embodiments are briefly described below. It should be understood that the drawings as described below merely illustrate some embodiments of the present disclosure, and should not be construed as limiting the scope of the present disclosure. Based on these drawings, other drawings can be obtained by those skilled in the art without inventive effort.

FIG. 1 is a flowchart of a driving state monitoring and feedback method based on multi-modal human-factor intelligent data analysis according to an embodiment of the present disclosure.

FIG. 2 is a flowchart of electroencephalographic data preprocessing according to an embodiment of the present disclosure.

FIG. 3 is a schematic diagram showing architecture of a driving state detection and early warning system according to an embodiment of the present disclosure.

FIG. 4 is a schematic flowchart of a driving state detection and early warning method according to an embodiment of the present disclosure.

FIG. 5 is a schematic flowchart of a training method for a cognitive distraction state identification model according to an embodiment of the present disclosure.

FIG. 6 is a schematic flowchart of another driving state detection and early warning method according to an embodiment of the present disclosure.

FIG. 7 is a flowchart of a fatigue detection method for a driver in a human-factor intelligent cabin according to an embodiment of the present disclosure.

FIG. 8 is a schematic diagram of driver fatigue state identification and monitoring according to an embodiment of the present disclosure.

FIG. 9 is a flowchart of a driver's driving state prediction method based on multi-modal data according to an embodiment of the present disclosure.

FIG. 10 is a schematic diagram of a driving state prediction system according to an embodiment of the present disclosure.

FIG. 11 is a schematic flowchart of preprocessing electroencephalographic data according to an embodiment of the present disclosure.

FIG. 12 is a flowchart of obtaining a target prediction result according to an embodiment of the present disclosure.

FIG. 13 is a flowchart of obtaining a first lateral velocity deviation value according to an embodiment of the present disclosure.

FIG. 14 is a flowchart of obtaining a predicted value of kinematic information according to an embodiment of the present disclosure.

FIG. 15 is a flowchart of obtaining a second lateral velocity deviation value according to an embodiment of the present disclosure.

FIG. 16 is a schematic structural diagram of a driving state monitoring and feedback system based on multi-modal human-factor intelligent data analysis according to an embodiment of the present disclosure.

### DETAILED DESCRIPTION OF THE EMBODIMENTS

To enable those skilled in the art to better understand the technical solutions of the present disclosure, description will be made clearly and completely on the technical solutions in the embodiments of the present disclosure with accompanying drawings. Obviously, the embodiments described below are only a part of the embodiments of the present disclosure, rather than all embodiments of the present disclosure. On a basis of the embodiments of the present disclosure, all other embodiments obtained by those skilled in the art without creative labor shall fall within the protection scope of the present disclosure.

It should be noted that terms such as “first” and “second” in the specification and claims of the present disclosure and in the accompanying drawings are intended to distinguish similar objects and do not necessarily describe a specific order or sequence. It should be understood that the numerals as used can be interchanged where appropriate, in such a manner that the embodiments of the present disclosure described herein can be implemented in an order other than those illustrated or described herein. In addition, terms “including” and “having” and any variations thereof are intended to cover non-exclusive inclusions. For example, a process, method, system, product, or server that includes a series of steps or units is not necessarily limited to those clearly listed steps or units, but may also include other steps or units that are not clearly listed or are inherent to the process, method, product, or device.

The present disclosure provides a driving state monitoring and feedback method and system based on multi-modal human-factor intelligent data analysis, and an edge computing terminal device. A driver state is identified through multi-modal human-factor data, and human-vehicle-road-environment monitoring and feedback is performed by combining multiple elements such as a driver, a vehicle, an environment, and a road condition.

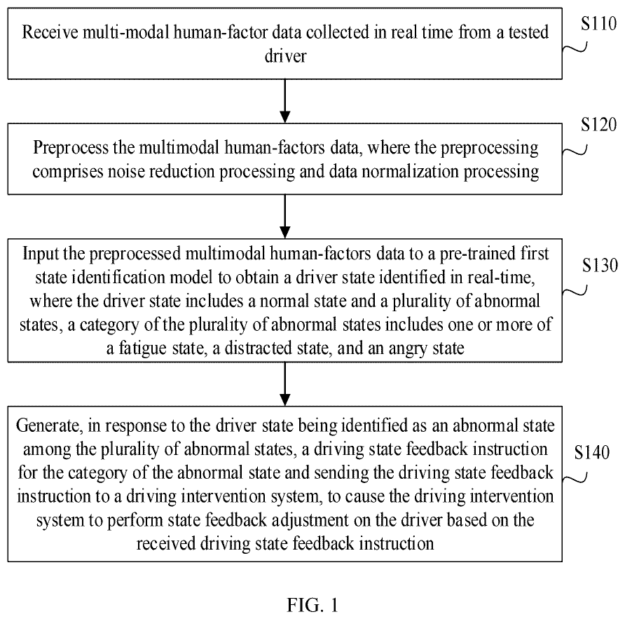

FIG. 1 is a flowchart of a driving state monitoring and feedback method based on multi-modal human-factor intelligent data analysis according to an embodiment of the present disclosure. The method includes the following steps S110 to S140.

At step S110, multi-modal human-factor data collected in real time from a tested driver is received. The human-factor data includes at least two of electroencephalographic data, heart rate data, electrodermal activity data, respiratory data, near-infrared data, blood oxygen data, blood pressure data, and skin temperature data.

At step S120, the multi-modal human-factor data is preprocessed. The preprocessing includes noise reduction processing and data normalization processing.

At step S130, the preprocessed multi-modal human-factor data is inputted to a pre-trained first state identification model to obtain a driver state identified in real-time. The driver state includes a normal state and a plurality of abnormal states. A category of the plurality of abnormal states includes one or more of a fatigue state, a distracted state, and an angry state.

In an implementation, there is a predetermined mapping relationship between human-factor data of different modalities and driver states with different labels. For example, signals from different regions or different frequency bands of the electroencephalographic data may be used to determine whether the driver is in the fatigue state, the distracted state, or the angry state, respectively; the heart rate data may be used to analyze whether the driver is in the angry state; the respiratory data may be used to analyze whether the driver is in the fatigue state; and the electrodermal activity data and near-infrared data may be used to analyze whether the driver is in the distracted state. The above are merely examples, and the present disclosure is not limited thereto.

At step S140, in response to the driver state being identified as an abnormal state among the plurality of abnormal states, a driving state feedback instruction is generated for the category of the abnormal state and is sent to a driving intervention system, to cause the driving intervention system to perform state feedback adjustment on the driver based on the received driving state feedback instruction.

In some embodiments of the present disclosure, the method further includes a pre-training process for the first state identification model. The first state identification model is pre-trained by: collecting baseline human-factor data and demographic data of the tested driver; retrieving, from a baseline state database, driver-related data having a similarity with the baseline human-factor data and the demographic data of the tested driver that is within a predetermined similarity range, and performing iterative training by using retrieved baseline human-factor data of the driver and driver state data stored in the baseline state database as a training set and using a pre-selected normal state and a plurality of pre-selected abnormal states as labels, to obtain the first state identification model.

In an exemplary embodiment of the present disclosure, an execution subject of the above-described method may be a vehicle computer. For example, the vehicle computer is connected to various collection devices, and these collection devices are worn by the driver or mounted inside the vehicle to collect the multi-modal human-factor data of the driver.

The driving state monitoring and feedback method and system based on multi-modal human-factor intelligent data analysis according to the present disclosure can process the pre-processed multi-modal human-factor data collected in real time by using the pre-trained first-state identification model, distinguish the normal state and abnormal state of the driver through classification labels, identify different driving states of the driver, and process for different driving states to avoid traffic accidents.

In some embodiments of the present disclosure, the noise reduction processing includes: applying one or more of wavelet filtering, Kalman filtering, and empirical mode decomposition to the electroencephalographic data; and performing noise reduction processing on the electrodermal activity data by using a value of a time window as a baseline value and a value of another time window as a maximum response value.

With this embodiment of the present disclosure, purer multi-modal human-factor data can be obtained.

In some embodiments of the present disclosure, preprocessing of the electroencephalographic data included in the human-factor data includes whole-brain average reference, filtering processing, ICA analysis, and time-frequency analysis to extract a target frequency band of the electroencephalographic data. The filtering processing is configured to screen the electroencephalographic data and remove artifact. The ICA analysis is configured to identify and remove a blink segment and/or an electromyographic segment. The time-frequency analysis is configured to extract the target frequency band of the electroencephalographic data. The filtering processing includes high-pass filtering processing, low-pass filtering processing, notch filtering processing, and band-pass filtering processing. The time-frequency analysis includes power spectral density analysis.

With this embodiment of the present disclosure, purer electroencephalographic data can be obtained.

In an embodiment of the present disclosure, the preprocessing of the electroencephalographic data included in the human-factor data further includes data cleaning prior to the whole-brain average reference. The data cleaning is configured to remove artifact and noise. The preprocessing of the electroencephalographic data included in the human-factor data further includes, prior to the ICA analysis, removing artifacts based on a deep learning method, and deleting a useless channel.

With this embodiment of the present disclosure, artifacts in the electroencephalographic data included in the human-factor data can be removed, and purer electroencephalographic data can be obtained, facilitating identification and feedback of the driving state of the driver.

In some embodiments of the present disclosure, the multi-modal human-factor data further includes image data of a head of the tested driver. The method further includes, subsequent to preprocessing the multi-modal human-factor data: analyzing, based on an image identification technology, the image data of the head of the tested driver to obtain an eye movement feature, a head movement feature, and a facial expression feature of the driver; and analyzing the eye movement feature and the head movement feature of the driver by using an eye tracking technology, and analyzing the facial expression feature by using an expression identification technology, to obtain the driver state identified in real-time.

With this embodiment of the present disclosure, a perspective for real-time identification of the driver state can be expanded, which can allow multi-perspective verification of the driver state, obtaining more accurate driver state identification result.

In some embodiments of the present disclosure, the method further includes, subsequent to preprocessing the multi-modal human-factor data: performing feature extraction on the preprocessed multi-modal human-factor data to obtain a driving behavior-related feature; and performing label management on the driving behavior-related feature; and performing, when data related to a label is detected, analysis based on the driving behavior-related feature to obtain the driver state identified in real-time. The driving behavior-related feature includes one or more of a heart rate, blood pressure, and a respiratory rate.

With this embodiment of the present disclosure, more refined management of the driver state can be achieved through label management, supporting more diverse driving state identification and feedback methods.

In some embodiments of the present disclosure, the first state identification model is a classification model. A type of the classification model includes any one of a vector machine model, a decision tree model, and a naive Bayes model.

In some embodiments of the present disclosure, the driver state further includes a cognitive state of the driver. The method further includes, subsequent to preprocessing the multi-modal human-factor data: obtaining operation data of the driver during driving, the operation data including a speed and a force of braking; obtaining an operation habit of the driver and setting a driver cognitive state identification threshold, based on historical operation data of the driver obtained through historical statistics; and determining that the cognitive state of the driver is abnormal in response to the operation data of the driver collected in real time during driving exceeding the driver cognitive state identification threshold.

With this embodiment of the present disclosure, the operation habit of the driver can be obtained by collecting and analyzing the historical operation data of the driver. Therefore, the abnormal driving state of the driver is identified by identifying abnormal operation conditions during driving operation, which provides a new perspective for identifying the driver state.

Further, based on the above driver state identification method, a weighted sum of the driver states obtained from different channels is obtained. When the weighted sum exceeds a threshold, the driver state identification result, i.e., the driving state, is generated.

In some embodiments of the present disclosure, the driving intervention system includes a voice module and a seat. The driving intervention system performing the state feedback adjustment on the driver based on the received driving state feedback instruction includes: (1) issuing a voice broadcast reminder or playing music, by using the voice module, to relive a mood of the driver; and (2) automatically adjusting a seat angle to remind the driver to adjust the driving state. It should be noted that, in a current intelligent driving system, many intelligent devices on the vehicle (such as a smart seat and a smart speaker) are connected to the vehicle computer, and the vehicle computer can issue instructions to these devices to implement pre-programmed functions, which is not limited in the present disclosure.

In some embodiments of the present disclosure, the demographic data includes at least two of an age, a gender, a height, and a weight of the driver, and the baseline state database uses a driver ID as a label and includes corresponding baseline human-factor data and driver state data. The above are merely examples, and the present disclosure is not limited thereto.

In some embodiments of the present disclosure, the method is implemented in a virtual driving environment simulated by a virtual reality technology. The virtual driving environment includes the tested driver and a driving vehicle, and further includes a pedestrian and other vehicles. The pedestrian and other vehicles respond to scene changes in the virtual driving environment.

In some embodiments of the present disclosure, when the virtual driving environment is a multi-task scenario, the multi-modal human-factor data includes eye tracking data. The method further includes: (1) calibrating a baseline of the collected multi-modal human-factor data; (2) performing extraction and training on the multi-modal human-factor data during measurement; and (3) identifying an eye-tracking region of interest based on an eye tracking technology, and extracting a relationship feature between the eye-tracking region of interest and a driving task scenario based on a SEEV model.

The virtual driving environment includes a driving operation cabin and a virtual reality road environment condition, and can simulate and construct any desired driving environment, thereby obtaining more accurate physiological information. In an embodiment of the present disclosure, to obtain more accurate physiological data, not only is data collection performed on the driver, but also reactions of different characters, such as real pedestrians and drivers of other vehicles, are added to the virtual environment. For example, in a scenario where a pedestrian is crossing an intersection and the driver stops the vehicle, and when a pedestrian-side traffic light is about to change in a few seconds, the physiological signal of the driver, including an electroencephalographic signal, an electromyographic signal, a heart rate, blood pressure, and a respiratory rate, etc., are collected to obtain a cognitive state of the driver corresponding to the current situation. In addition, physiological information and movement information of the pedestrian are detected, such as a gesture to interact with the driver and signal “I want to cross the road, please wait a moment.” Also, a physiological signal, an emotional state, and other information of the driver are monitored to determine a driving state and habit of the driver. This is merely an example of a scenario, which is not specifically limited in the present disclosure.

Further, when the driving task scenario is the multi-task scenario, the relationship feature between the region of interest and the driving task scenario is extracted based on the SEEV (salience S, effort E, expectancy E, and value V) model. The relationship feature includes: importance of the driving task scenario (task priority), relevance between the region of interest and the driving task scenario (relevance), and an amount of information in the region of interest (bandwidth, BW). These features are further combined into visual attention to different areas of interest (AOI) in a rule base under the driving task scenario, establishing the rule base stored in a form of (scenario, AOI, and visual attention) triples. The visual attention to different areas of interest is expressed as:

$$Visual Attention ( Scan ) to A O I = {{\sum }_{tasks}}_{[ ( B W * relevance ( value ) of A O I to task * task priority ]} . \qquad ( 1 )$$

With this embodiment of the present disclosure, a distracted state of the driver can be verified from another perspective based on the eye movement data and eye tracking technology.

In the above embodiments of the present disclosure, it is further necessary to calibrate the baseline of the collected multi-modal human-factor data to perform extraction and training on eye movement and physiological features during measurement. The measurement process is as follows. A feature-level physiological indicator is generated from the human-factor data through discriminant analysis, and the feature-level physiological indicator is represented by binary classification data to indicate whether the driver state is normal or abnormal. A significant change in the feature-level physiological indicator is obtained, and the significant change may be set as a mean value within 4 seconds±a standard deviation, or may be custom-set to other values, which are merely examples, and the present disclosure is not limited thereto. The discriminant analysis can perform discriminant analysis on the human-factor data by using a Bayesian decision theory, a linear discriminant function, a nonlinear discriminant function, and a support vector machine.

Further, when the driver state is in normal, a feature-level eye movement indicator is generated from the eye movement data based on the constructed rule base. A first evaluation score of the driver in the driving task scenario is obtained by matching the feature-level eye movement data with a theoretical degree of the eye-tracking region of interest extracted from the rule base. For example, the first evaluation score is obtained by a correlation coefficient method, which is used to match the eye movement data in the region of interest with the theoretical attention degree in the rule base. For example: σ1(A1, A′1)=0.76, σ2(A2, A′1)=0.96, σ3(A3, A′1)=0.27; where A′1 represents a theoretical attention vector to different regions of interest, A1, A2, and A3 represent vectors of feature-level eye movement indicators in the different regions of interest, respectively, and σ1, σ2, and σ3 represent first evaluation scores in the different regions of interest, respectively, which are further used to determine cases of inattention.

Based on information obtained from the human-vehicle-road-environment loop, vehicle data is encoded by using a Transformer Encoder, and two fully connected (FC) layers are added subsequent to the Transformer Encoder to ensure that the model output has the same length and dimension as the input data. A training process of a feature extraction model is as follows. For input vehicle information of multiple types, random masking with a proportion of 0.1 is performed in the time dimension, and random masking is also performed in a channel dimension, to mask information of one or two channels. The masked data is input into the Transformer Encoder and FC layers, and the output result is compared with the unmasked input data to calculate a loss and perform backpropagation. After the training is complete, the output of the model can fully recover the masked parts in the input data. During this process, the Transformer Encoder learns effective features in the data. These features are combined with features from other modal data, such as the human-factor data, emotional data, cognitive data, and environmental data, for classification tasks. The classification tasks include, but are not limited to, the driving state, habit, etc. Monitoring is performed based on the obtained driving habit and driving state, and feedback information is provided.

Driving operation feedback is performed based on the detected emotional state. The emotional state affects attention and alertness of the driver. When the driver is in a positive emotional state, the attention and alertness are enhanced, making the driver more likely to detect a dangerous and emergency situation on the road and respond more quickly. Conversely, when the driver is in a negative emotional state, the attention and alertness are decreased, potentially making the driver less sensitive to the condition on the road and leading to a slow response or misjudgment. Therefore, when the driver is monitored to be in a low mood, prompt reminders are provided to improve concentration, or a positive response is given to help lift the mood of the driver to a certain extent.

In addition, the emotional state affects a decision-making ability of the driver. That is, the emotional state has a significant impact on the decision-making ability of the driver. When is in the positive emotional state, the driver is more likely to make a wise and rational decision and has a more accurate judgement about the driving behavior and road condition. Conversely, when in the negative emotional state, the driver may make an impulsive decision and has an incorrect judgement about the driving behavior and road condition, increasing a risk of traffic accidents.

Since the emotional state affects the driving behavior of the driver, it affects the driving behavior and operation of the driver. When in the positive emotional state, the driver is more likely to maintain a stable driving behavior, comply with traffic rules and instructions, and control the vehicle more accurately. Conversely, when in the negative emotional state, the driver may exhibit an unstable driving behavior, violate the traffic rules and instructions, and control the vehicle less accurately, increasing the risk of traffic accidents.

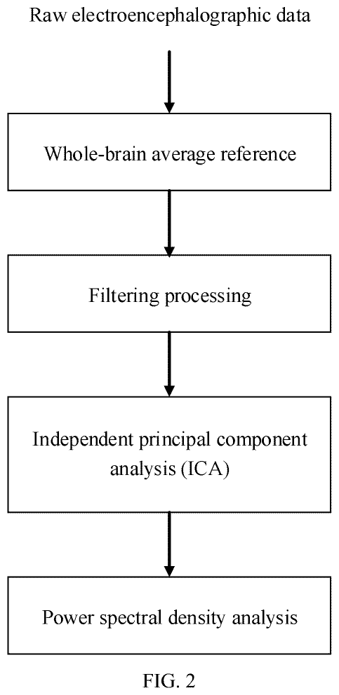

The processing of raw electroencephalographic data in the present disclosure is relatively complex and involves multiple more detailed steps. FIG. 2 is a flowchart of electroencephalographic data preprocessing according to an embodiment of the present disclosure. Whole-brain average reference is performed on the obtained raw electroencephalographic data. Filtering processing is then performed to remove a less necessary frequency band and retain a frequency band important for driving state analysis. Next, independent principal component analysis is performed, and power spectral density analysis may be performed to extract a target frequency band of the electroencephalographic data for driving state identification.

In an embodiment of the present disclosure, the raw electroencephalographic data is subjected to the whole-brain average reference and a band-stop filtering with a frequency range of 0.5 Hz to 45 Hz. The blink segment and the electromyographic segment are identified and removed through ICA principal component analysis. A power spectral density feature including a theta wave of 3 Hz to 7 Hz, an alpha wave of 8 Hz to 12 Hz, and a beta wave of 13 Hz to 30 Hz is extracted through the time-frequency analysis.

Basic steps of electroencephalographic data preprocessing include: (1) locating a channel position: a coordinate position on the scalp is assigned to each channel based on an electrode arrangement system to facilitate spatial analysis and visualization, (2) removing useless channels, and (3) setting a threshold to remove some segments containing excessive artifacts or outliers, to improve data quality.

During locating the channel position, a clean signal is distinguished from artifacts and noise by mathematical cleaning. Selecting a reasonable filter based on frequency ranges of the artifacts can effectively reduce the artifacts of the raw electroencephalographic data. For example, a high-pass filter is set to 0.1 Hz and a low-pass filter is set to 30 Hz. In addition, to eliminate interference from the mains power supply, a notch filter may be selected to remove 50 Hz interference. Also, when a certain signal frequency range is of interest, a bandpass filter may be used. For example, if the alpha wave is of interest, the bandpass filter may be used to retain the signal frequency band of 8 Hz to 13 Hz for analysis.

During the removing of the useless channels, some unnecessary channels, such as bilateral mastoid points and electrooculographic channels, are removed based on the experimental objective and design. Other multi-modal biological data may be combined to help detect and distinguish artifact data in the electroencephalographic data. For example, electrooculographic and eye movement measurement is combined to identify the blink and eye movement in the EEG signal, electroencephalographic signal measurement is combined to identify electroencephalographic artifacts in the EEG signal, and acceleration signal measurement is combined to identify head movement artifacts in the EEG signal. A plurality of electrodes are configured as a single channel that is referred to a single lead. The layout typically includes different channel configurations such as 16-lead and 32-lead. If some electrodes within the single lead are not accurately placed on the scalp, a brain neurophysiological signal cannot be accurately collected, and the single lead is classified as a bad lead. Causes of the bad lead include channel failure, incorrect electrode placement, poor contact, series connection, and channel saturation. During data analysis, a bad lead is identified and removed. A signal of the bad lead is filtered out through typically comparing with normal electrode signals in other channels.

When setting the threshold to remove some segments containing the excessive artifacts or outliers, data preprocessing typically involves removing a part of noise by setting a threshold, identifying abnormal frequency bands. Selecting the reasonable filter based on the frequency ranges of the artifacts can effectively reduce the artifacts of the raw electroencephalographic data. For example, the high-pass filter is set to 0.1 Hz and the low-pass filter is set to 30 Hz. In addition, to eliminate the interference from the mains power supply, the notch filter may be selected to remove the 50 Hz interference. Also, when a certain signal frequency range is of interest, the bandpass filter may be used. For example, if the alpha wave is of interest, the bandpass filter may be used to retain the signal frequency band of 8 Hz to 13 Hz for analysis. After considering all of the above-described factors, the collected EEG signal may still contain much noise, for which the filter and mathematical algorithm may be further used to separate the signal from the noise, improving the signal-to-noise ratio.

Further, an A1 algorithm may be trained by using an existing data set based on the deep learning method for automatic detection and rejection of artifacts in recordings (ideally in real time), to reduce a mean-square error between a target signal and a predicted signal.

A commonly used algorithm flow for data preprocessing is as follows.
(1) Data segmentation, for example, a 0.5 s time window is segmented as an input signal.
(2) Fast Fourier transform is performed on the EEG signal to extract its frequency domain information.
(3) Distribution of EEG electrode points in a three-dimensional space is projected to a two-dimensional image using azimuthal equidistant projection in the Cartesian coordinate system, converting the EEG signal into a multispectral image with a spatial topological structure.
(4) Information of the EEG signal in the time dimension, frequency domain dimension, and spatial dimension can be fully explored through the above-described three steps.

In addition, based on the above preprocessing steps, methods such as SNS, RANSAC, and Cross-Validation may also be used to further improve the signal noise ratio (SNR) of the electroencephalographic signal to a certain extent.

Subsequent to obtaining the electroencephalographic signal, further analysis may be performed based on a brain topographic map to determine which frequency band of the brain has a higher degree of activation, and which brain region of the brain the frequency band with the higher degree of activation is located within, within a predetermined time period. For example, when the tested driver is exposed to visual stimuli of scary images, it is observed that a brain topographic map of the Beta wave is more activated, reflects a state of tension, and is located in the visual cortex of the occipital lobe (the rear part of the brain) region. Different electroencephalographic signals and different locations where these electroencephalographic signals occur determine a mode, a perceptual ability, a distraction condition, a fatigue condition, and a workload level of the tested driver at the current moment, giving different responses during actual driving.

On the basis of identifying the driving state using a state video model as described in steps S130 to S140, the method may further include identifying the driving state based on a driving behavior through an image identification technology. For example, changes in the psychological activity of the driver may be explored through an eye movement characteristic, a head movement characteristic, and a facial expression of the driver, determining whether the driver is experiencing driving fatigue, driving distraction, or other states. With this method, it can be determined whether the driver is engaged in distracted driving or fatigued driving by identifying and analyzing the eye feature. The eye movement feature and a rotation angle are extracted by using a neural network algorithm, and by combining angles between two sides of rearview mirrors of different vehicles and a position of the driver, it is determined whether the driver is normally observing the rearview mirror during driving or is distracted.

In an exemplary embodiment of the present disclosure, the driving state may be determined by combining the obtained eye movement data, a relationship between the eye rotation angle and a set angle range of the rearview mirror, and the electroencephalographic signal, thereby performing intervention for the driver state.

In an embodiment, the driving state may be identified based on the driving behavior through the eye tracking technology. In an exemplary embodiment of the present disclosure, it may be determined whether the driver is engaged in distracted driving or fatigued driving by tracking a gaze direction and a gaze point of the driver. With this method, it can be determined whether the driver is gazing at the road ahead by analyzing the eye movement and gaze direction of the driver, thereby determining whether the driver is engaged in distracted driving or fatigued driving.

In an embodiment of the present disclosure, the driving state may be identified based on the physiological signal. For example, it may be determined whether the driver is engaged in distracted driving or fatigued driving by monitoring the physiological signal, such as a heart rate and blood pressure of the driver. With this method, it can be determined whether the driver is fatigued or distracted through analysis of the physiological signal of the driver, which is achieved by comparing a predetermined threshold with a baseline value.

Further, the deep learning algorithm model for obtaining the driving behavior based on the physiological signal may adopt a feature extraction algorithm configured to extract the driving behavior-related features from the obtained physiological signal data of the driver. These features may include a heart rate, blood pressure, a respiratory rate, etc. Label management is performed on these extracted features. When data related to a label is detected, the driving state and emotional state of the driver is determined by analyzing these data features (carrying the labels). When the driving state and emotional state of the driver are obtained, classification management is performed to form a database, and different driving habits are set correspondingly. When the driving habit does not meet a set driving habit level, a reminder is issued along with an improvement plan.

The deep learning algorithm model may be a classification algorithm model. The extracted features are classified into different driving behaviors, such as normal driving, distracted driving, and fatigued driving based on the classification algorithm model. Common classification algorithms include a support vector machine, a decision tree, naive Bayes, etc.

In an embodiment of the present disclosure, since the cognitive state of the driver affects the driving behavior and decision-making ability, such as judgment and reaction speed of the driver, the driving ability and state of the driver may be determined by evaluating the cognitive state of the driver, which requires obtaining data during operations of the driver, including the speed and force of braking, to calculate the driving habit.

Further, in an embodiment of the present disclosure, the driving state may be evaluated from three dimensions: the driver state, a vehicle state, and a road environment state, which helps to improve accuracy of driving state evaluation, issue a risk early warning based on the driving state, and enhance driving safety.

A driving state detection and warning method according to an embodiment of the present disclosure is described with reference to FIG. 4 to FIG. 6.

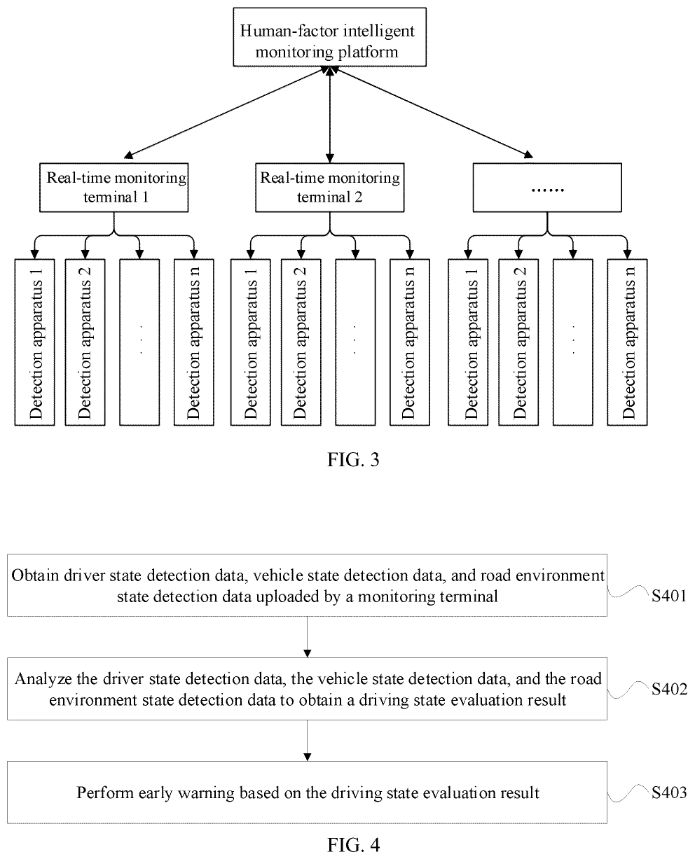

FIG. 3 is a schematic diagram showing architecture of a driving state detection and early warning system according to an embodiment of the present disclosure. As illustrated in FIG. FIG. 3, the driving state detection and warning system includes a human-factor intelligent monitoring platform and a real-time monitoring terminal. The human-factor intelligent monitoring platform can monitor a single real-time monitoring terminal or simultaneously monitor a plurality of real-time monitoring terminals. When the real-time monitoring terminal is activated, the human-factor intelligent monitoring platform and the real-time monitoring terminal achieve information intercommunication via a network protocol such as a Hypertext Transfer Protocol (HTTP) or a Transmission Control Protocol (TCP). The real-time monitoring terminal uploads terminal information to the human-factor intelligent monitoring platform, and the human-factor intelligent monitoring platform matches the uploaded terminal information with terminal information pre-stored on the platform, to determine a successfully matched monitoring terminal. The human-factor intelligent monitoring platform can perform real-time visual presentation and processing on an early warning state of the successfully matched real-time monitoring terminal, and can also conduct visual presentation, as well as data statistics and analysis on historical information of the early warning state of the real-time monitoring terminal.

Each real-time monitoring terminal includes a plurality of detection apparatuses, including a physiological signal detection device, a video signal detection device, a vehicle signal detection device, and a motion capture detection device, and so on. The physiological signal detection device is configured to detect multi-modal physiological data, which refers to state data of the driver collected through a plurality of sensors. These sensors may be physiological sensors that obtain the physiological data of the driver during driving, such as an eye movement tracking device, an electrodermal activity sensor, an electroencephalographic sensor, a respiratory sensor, and an electroencephalographic sensor. Data collected by the eye movement tracking device includes, but is not limited to, a pupil diameter, a blink frequency, a fixation duration, and the number of fixations of eye movement of the driver. The detection apparatus is configured to detect the driver state, the vehicle state, and the road environment state. The real-time monitoring terminal may activate the plurality of detection apparatuses simultaneously with a single button, or activate each of the plurality of detection apparatuses individually. Collection sensors for the multi-modal physiological data may be integrated into a single terminal device or may be combined to form a system. The terminal device integrated with the plurality of collection sensors may be deployed in a local driving vehicle. In another embodiment, the collection sensors may be mounted in the local vehicle, and the terminal device performing the analysis and processing is disposed at a server side.

The real-time monitoring terminal detects the signal and sends the to a cloud server. The cloud server may determine whether the detected signal is a normal signal, that is, whether the signal is a high-quality and valid signal, through the neural network algorithm. When the signal is not the normal signal, the driver is prompted to perform apparatus adjustment via voice or an image to obtain the normal detection signal. Subsequent to the real-time monitoring terminal being connected to the detection apparatus, to ensure quality of data presentation, the raw data is stored in the local client, and is uploaded to the cloud server subsequent to completing monitoring to form a historical database.

FIG. 4 is a schematic flowchart of a driving state detection and early warning method according to an embodiment of the present disclosure. The method includes following steps S401 to S403.

At step S401, driver state detection data, vehicle state detection data, and road environment state detection data uploaded by a monitoring terminal are obtained.

In this embodiment, the plurality of detection apparatuses obtain the driver state detection data, the vehicle state detection data, and the road environment detection data of the driver during driving. Subsequent to the monitoring terminal being connected to the detection apparatuses, the monitoring terminal uploads these data to the human-factor intelligent monitoring platform. The driver state detection data may not only include multi-modal physiological data, but also include other behavior data of the driver during driving, data reflecting an emotion or a feeling of the driver, and data used to determine a perceptual ability of the driver. The data for determining the perceptual ability may be obtained by detecting feedback of the driver during driving through different operations. The driver state during driving is determined through the above detected data.

In some embodiments, the driver state includes a fatigue state, a distracted state, and an emotional state.

In an embodiment of the present disclosure, the fatigue state includes mild fatigue, moderate fatigue, and severe fatigue, and is obtained by analyzing data detected by the physiological signal detection device or the video signal detection device. For example, the electroencephalographic data or power spectral density (PSD) data are obtained through electroencephalogram (EEG) detection, or changes in a pupil state or a blink state of the driver are obtained through detection of an in-vehicle camera, and a fatigue level of the driver during driving is obtained by analyzing these data or changes in states.

In some embodiments, the distracted state includes behavioral distraction and cognitive distraction. The behavioral distraction includes making or answering a phone call, smoking, chatting, and failing to look ahead for a long duration, and the cognitive distraction includes low-level distraction, moderate-level distraction, and high-level distraction.

In an embodiment of the present disclosure, the behavioral distraction is obtained by analyzing the data detected by the video signal detection device. For example, dangerous driving behavior of the driver caused by distraction is detected through a behavioral distraction state detection device (the in-vehicle camera, a video monitor, etc.). The cognitive distraction is obtained by analyzing the data detected by the physiological signal detection device. For example, data such as heart rate variability (HRV), heart rate (HR), inter-beat interval (IBI, i.e., R-R interval), and skin conductance response (SCR) is obtained through a photoplethysmography (PPG) wireless pulse sensor or an electrodermal activity (EDA) wireless electrodermal activity sensor, and the level of cognitive distraction is obtained by analyzing these data.

In some embodiments, the emotional state includes emotional intensity and emotional valence. The emotional intensity includes emotion abnormality and emotion normality. The emotional valence includes a positive emotion and a negative emotion. The positive emotion refers to positive feelings such as happiness and excitement, while the negative emotion refers to negative feelings such as anger, irritation, and sadness.

In an embodiment of the present disclosure, the emotional intensity is obtained by analyzing the data detected by the physiological signal detection device. For example, data such as the heart rate variability (HRV), heart rate (HR), inter-beat interval (IBI, i.e., R-R interval), and skin conductance response (SCR) is obtained and analyzed through the photoplethysmography (PPG) wireless pulse sensor or the electrodermal activity (EDA) wireless electrodermal activity sensor, and the level of cognitive distraction is obtained by analyzing these data. Exemplarily, whether the emotion is abnormal is determined based on a change in a skin conductance response (SCR) value per minute. The emotional valence is obtained by analyzing the data detected by the video signal detection device (such as a facial expression detection device). Exemplarily, whether the emotional valence of the driver is positive or negative is determined by detecting a change in the facial expression of the driver.

In some embodiments, the vehicle state includes a normal driving state and an abnormal driving state, and is obtained by analyzing the data detected by the vehicle signal detection device. In an embodiment of the present disclosure, operation speed of the vehicle, steering wheel data, pedal data, global positioning system (GPS) data, etc. are detected by Vehub, and the collected data is analyzed, including lateral acceleration analysis, center of mass sideslip angle constraint analysis, friction circle constraint analysis, tire sideslip angle analysis, built-in advanced driving performance analysis and so on, to determine whether the vehicle is in the normal driving state.

Further, real-time coding and post-hoc coding may be performed on the driving behavior to analyze and count the behavior. The driving behavior includes pedal operations (braking, accelerating), steering behavior (turning left, turning right), lane change behavior (changing to the left lane, changing to the right lane, reversing, making a U-turn, driving in the right lane, driving in the left lane), driving speed (high speed, medium speed, low speed, parking or idling), longitudinal acceleration (strong acceleration, normal acceleration, weak acceleration), and steering wheel operations (clockwise, counterclockwise), determining whether the vehicle is in the normal driving state. For example, whether the driver frequently brakes suddenly may be determined by analyzing the pedal operations. If the driver frequently brakes suddenly, it indicates that the currently driven vehicle is in the abnormal driving state.

In some embodiments, the road environment state includes no vehicle or pedestrian ahead, vehicle collision ahead, lane departure, excessive proximity to other vehicles, frequent lane changes, another vehicle cutting in, speeding vehicles ahead, pedestrian collision, etc. The vehicle collision ahead, pedestrian collision, excessive proximity to other vehicles, and frequent lane changes each include presence and absence. The lane departure includes leftward departure or rightward departure. A level of speeding includes low, medium, and high. The other vehicle cutting in includes left-side cutting-in and right-side cutting-in.

In an embodiment of the present disclosure, the road environment state is detected and obtained by an Advanced Driving Assistance System (ADAS). In an embodiment of the present disclosure, the road environment state may further include environmental data obtained by a third party, such as weather, temperature, and humidity.

At step S402, the driver state detection data, the vehicle state detection data, and the road environment state detection data are analyzed to obtain a driving state evaluation result.

In an embodiment of the present disclosure, the driver state detection data, the vehicle state detection data, and the road environment state detection data are input into corresponding state identification models, respectively, to identify and obtain the driver state, a vehicle state, and a road environment state; and the driver state, the vehicle state, and the road environment state are combined to obtain the driving state evaluation result.

In an embodiment of the present disclosure, in response to the driver state being mild fatigue, the vehicle state being a normal driving state, and the road environment state being no vehicle or pedestrian ahead, the driving state evaluation result is a first driving risk state; in response to the driver state being moderate fatigue, the vehicle state being an abnormal driving state, and the road environment state being no vehicle or pedestrian ahead, the driving state evaluation result is a second driving risk state; and in response to the driver state being severe fatigue, the vehicle state being the abnormal driving state, and the road environment state being another vehicle cutting in, the driving state evaluation result is a third driving risk state.

It should be noted that, the present disclosure may also provide other indicators for evaluating the driving state. The driving state evaluation may also be performed based on the driver state detection data and the vehicle state detection data, or based on the vehicle state detection data and the road environment state detection data, or based on the driver state detection data and the road environment state detection data. For example, the driving evaluation result is a fourth driving risk state in response to the driver state being mild fatigue and the vehicle state being the abnormal driving state. The driving evaluation result is a fifth driving risk state in response to the vehicle state being the abnormal driving state and the road environment state being lane departure. The above is only for illustrative purposes, and the present disclosure does not limit division of risk levels or evaluation indicators of the driving state.

In some embodiments, the obtained multi-modal data (including the driver state detection data, the vehicle state detection data, and the road environment state detection data) is input into a convolutional neural network model to obtain a multi-modal matrix through mapping. The convolutional neural network model has a 3*3 structure. The multi-modal matrix is imported into a Transformer model to calculate a correlation weight between different data elements in the multi-modal discrete data, i.e., to obtain the correlation weight between the multi-modal data, and the driving state evaluation result is obtained by combining the correlation weight.

In an embodiment of the present disclosure, by using this 3×3 convolutional neural network, the number of discrete data may be mapped into an m-dimensional vector. This vector may be regarded as a feature representation of the discrete data, with each dimension corresponding to a feature of the discrete data. Assuming n pieces of discrete data are provided, a 3n×3m-dimensional multi-modal raw matrix is obtained by processing these data through the 3×3 convolutional neural network. In this matrix, each row represents the feature representation of one discrete data, while each column represents a different feature. The multi-modal raw matrix retains feature information of the discrete data, and the discrete data is converted into a vectorized form through feature extraction and mapping of the convolutional neural network. The multi-modal raw matrix is imported into the Transformer model, and correlation calculation is performed based on a self-attention mechanism in the Transformer model. The self-attention mechanism allows the model to analyze each element in the input sequence and adjust weights based on interrelationships among the elements, and the driving state evaluation result is obtained by combining the correlation weights.

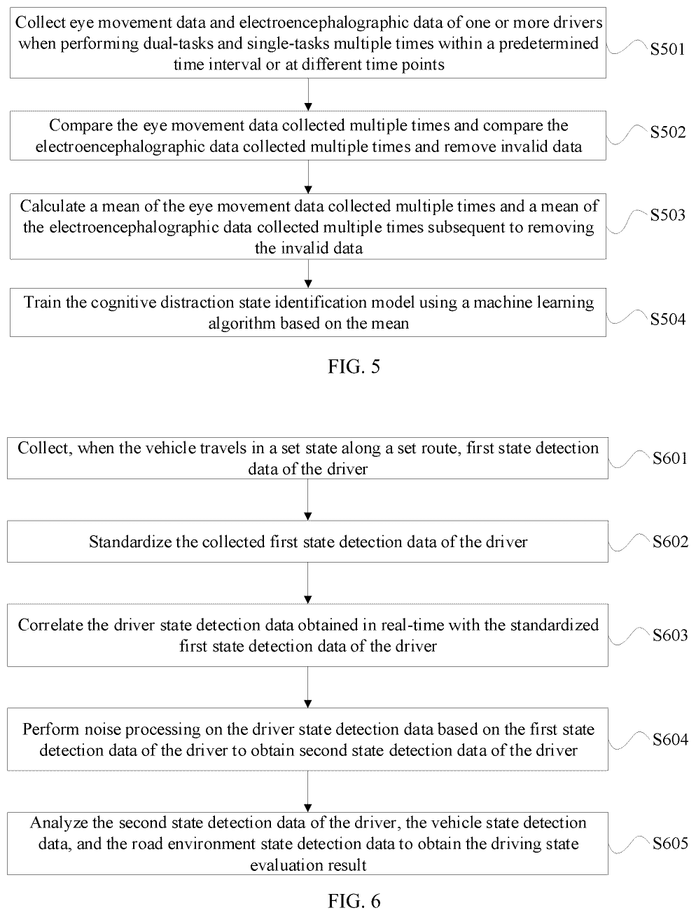

In some embodiments, the state identification model includes a driver state identification model. The driver state identification model includes a cognitive distraction state identification model. As shown FIG. 5, which is a schematic flowchart of a training method for a cognitive distraction state identification model according to an embodiment of the present disclosure, the training method for a cognitive distraction state identification model includes the following steps S501 to S504.

At step S501, eye movement data and electroencephalographic data of one or more drivers when performing dual-task and single-task are collected multiple times within a predetermined time interval or at different time points.

In an initial stage of model establishment, it is necessary to accumulate a large amount of data under different scenarios to train the model. In the present disclosure, eye movement data and electroencephalographic data of a single driver when performing the dual-task and the single-task, as well as eye movement data and electroencephalographic data of a plurality of different drivers when performing the dual-task and single-task are collected, respectively. Also, a plurality of collections and recordings are performed to ensure completeness of data.

Further, a plurality of data collections at different time periods or time points may be performed for one driver or a plurality of different drivers. For example, data may be collected within the predetermined time interval or at different time points. For example, data is collected from 8:00 to 11:00, 14:00 to 17:00, or at 6:00, 10:00, 13:00, 15:00, 19:00, 24:00 (midnight), and 2:00 a.m., and so on. Data under different states is recorded based on different time periods or time points. Because each driver exhibits different cognitive distraction states at different time periods or time points, collecting data from different time periods or time points for training during model training can increase the completeness and accuracy of the data, ensure the reliability of the model, and improve the accuracy of driving state evaluation, thereby further enhancing driving safety.

At step S502, the eye movement data collected multiple times is compared, and the electroencephalographic data collected multiple times is compared and invalid data is removed.

In an embodiment of the present disclosure, by comparing the data collected multiple times and removing the invalid data, such as abnormal data or data with large deviations, the accuracy of the data can be improved and the amount of data processing is reduced.

At step S503, a mean of the eye movement data collected multiple times and a mean of the electroencephalographic data collected multiple times subsequent to removing the invalid data are calculated.

In an embodiment of the present disclosure, subsequent to removing the invalid data, a mean of remaining data is calculated and saved.

At step S504, the cognitive distraction state identification model is trained using a machine learning algorithm based on the mean.

However, erroneous determination of the distracted state is prone to occur during a driver distraction state identification process. For example, when driving normally, the driver may need to turn the head or eyes to check left or right rearview mirror for driving safety, or the heart rate or electroencephalographic data of the driver may change due to vehicle jolting, which causes the system to erroneously determine that the driver is in the distracted state during driving.

Further, as illustrated in FIG. 6, which is a schematic flowchart of a driving state detection and early warning method according to another embodiment of the present disclosure, the method includes the following steps S602 to S605.

At step S601, first state detection data of the driver is collected when the vehicle travels in a set state along a set route.

The vehicle travels in the set state along the set route, where the set state refers to a normal driving state of the driver, for example, a compliant driving state where the driver has no distracted behavior, such as making a phone call, smoking, chatting, and failing to look ahead for a long duration, and no fatigue. In this case, the first state detection data of the driver, that is, detection data of the normal driving state of the driver, is collected. The first state detection data of the driver includes at least a physiological signal, a first eye movement signal, an electroencephalographic signal, a brain imaging signal, and behavioral detection data of the driver.

At step S602, the collected first state detection data of the driver is standardized.

In an embodiment of the present disclosure, the collected first state detection data of the driver is converted into a predetermined unified format to maintain the data within a certain range. For example, the heart rate is converted into a data range format, and all heart rate values within this range belong to the normal driving state of the driver. Standardization of the first state detection data of the driver facilitates eliminating differences in attribute features such as property, dimension, and order of magnitude between different detection data, thereby converting the data into dimensionless standardized values.

At step S603, the driver state detection data obtained in real time is correlated with the standardized first state detection data of the driver.

At above step S401, driver state detection data of other tested drivers during driving, including the multi-modal physiological data (including the electroencephalographic data, the eye movement data, the heart rate, etc.), the behavior data, etc., are collected in real time by the plurality of detection apparatuses. The driver state detection data collected at step S401 is compared and correlated with the first state detection data of the driver collected at step S601 to determine which parts of the driver state detection data fall within the detection data range of the normal driving state.

At step S604, noise processing is performed on the driver state detection data based on the first state detection data of the driver to obtain second state detection data of the driver.

In some embodiments, the driver state detection data includes a second eye movement signal obtained by parsing video content obtained in real time by the video signal detection device. Artifact and noise in the second eye movement signal is determined, that is, the eye movement signal in the second eye movement signal that belongs to the normal driving state but is erroneously determined as the distracted state is determined, in combination with the first eye movement signal in the first state detection data of the driver. The artifact noise is removed to obtain the second state detection data of the driver, i.e., to obtain the driver state detection data subsequent to removing the artifact noise.

In an embodiment of the present disclosure, the noise processing may also be performed on other data in the driver state detection data, such as the heart rate, the electroencephalographic signal, and the behavior data.

At step S605, the second state detection data of the driver, the vehicle state detection data, and the road environment state detection data are analyzed to obtain the driving state evaluation result.

In the embodiment of the present disclosure, by pre-obtaining the normal driving state data of the driver and performing data standardization, the collected driving state detection data is compared and correlated with the standardized normal driving state data. The noise processing is performed to identify and remove the artifact noise, eliminating the effect of erroneous determination of the distracted state and improving the accuracy of driving state evaluation.

On the basis of the driving state evaluation result obtained at step S402 or step S605, the above step S403 is further executed, i.e., the early warning is performed based on the driving state evaluation result.

In an embodiment of the present disclosure, the human-factor intelligent monitoring platform reminds or warns the driver using voice or a visual image based on the driving state evaluation result. For example, if the driver experiences driving fatigue due to long-term driving, the human-factor intelligent monitoring platform can remind the driver in a form of voice or the image: “You have been driving for a long time, please stop and rest.” For example, when the driver is distracted by making or answering a phone call while driving at a high speed in a complex surrounding traffic condition, the intelligent human-factor monitoring platform can issue an alarm to the vehicle, to remind the driver to pay attention to the vehicle ahead.

Further, a detrimental driving habit is marked based on the driver state detection data. The detrimental driving habit includes smoking, making a phone call, cutting in line, failing to look ahead for a long duration, frequent sudden braking, frequent speeding, etc. In addition, the number of occurrences of the detrimental driving habit is counted. When the number of occurrences of the detrimental driving habit exceeds a predetermined number (e.g., three), the driver is reminded or warned. In another embodiment of the present disclosure, for a vehicle equipped with an autonomous driving system, when it is determined that the operation of surrounding vehicles is safe, the vehicle may be pulled over by controlling the steering wheel, brake, and turn signal.

In addition, by collecting the driver state detection data, the vehicle state detection data, the road environment state detection data, human-machine interaction data (such as human machine interface (HMI) human-machine interaction data and head-up display (HUD) human-machine interaction data), electroencephalographic data, eye movement data, etc., a basic ability of the driver such as alertness, attention span, and memory can be evaluated, or a special ability of the driver such as a spatial orientation ability, motor coordination ability, and peripheral visual field level is evaluated, or a personality trait of the driver is evaluated, or driving suitability is evaluated. Also, the system may further construct a cognitive ability data profile of the driver, providing data support for evaluation, training, and selection of driver abilities.

In the embodiments of the present disclosure, the driver state detection data, the vehicle state detection data, and the road environment state detection data are simultaneously detected and obtained through the plurality of different detection apparatuses, a comprehensive analysis is performed on the driver state detection data, the vehicle state detection data, and the road environment state detection data to obtain the driving state evaluation result, and the early warning is performed based on the driving state evaluation result. The present disclosure evaluates the driving state from three dimensions: the driver state, the vehicle state, and the road environment state, which helps to improve the accuracy of driving state evaluation and improve driving safety. Also, the driver state, the vehicle state, and the road environment state are classified into different levels. For example, the driving state is divided into the fatigue state, the distracted state, and the emotional state, and the fatigue state, distracted state, and emotional state are further subdivided, further improving the accuracy of the driving state evaluation. A risk early warning is performed based on the driving state, improving the driving safety.

Further, in the embodiments of the present disclosure, at least one independent variable feature of the driver may further be extracted based on the collected physiological data and behavior data of the driver, and an actual fatigue state of the driver is identified using the at least one independent variable feature and the vehicle is controlled to issue a state reminder for the driver, improving the accuracy of fatigue state identification of the driver, increasing the reliability of the identification result, enhancing driver experience and customer stickiness, and meeting different needs of the driver in different scenarios. In addition, it solves the problem that the driving state of the driver is detected solely based on the facial expression of the driver and road information, which results in a relatively simple fatigue detection method and identification result, failing to accurately identify the actual driving state of the driver and reducing the accuracy and reliability of the detection result, and results in a narrow scope of application, failing to meet the actual detection need of the driver and reducing usage experience of the driver.

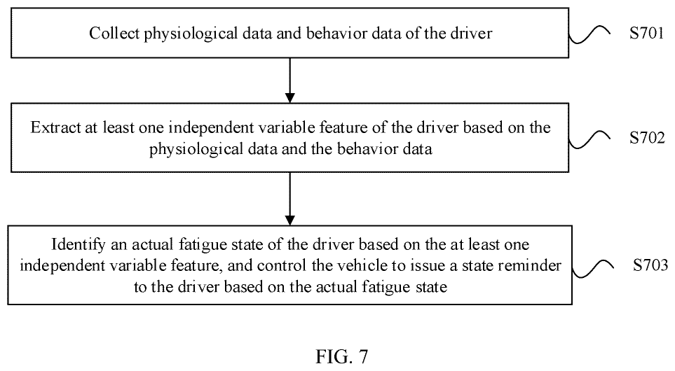

In an embodiment of the present disclosure, FIG. 7 is a flowchart of a method for detecting fatigue of a driver in a human-factor intelligent cabin according to an embodiment of the present disclosure.

As illustrated in FIG. 7, the method for detecting fatigue of the driver in the human-factor intelligent cabin includes the following steps S701 to S703.

At step S701, physiological data and behavior data of the driver are collected.

It should be understood that, the physiological data herein refers to a human body's physiological signal relevant to identifying the driver state, such as body temperature, a heart rate, and blood pressure. The behavior data herein refers to data of behavior relevant to identifying the driver state, such as a head posture or a hand movement.

In the embodiments of the present disclosure, the physiological data and the behavior data of the driver may be collected. Because physiological and behavior indicators of the driver undergo certain changes during long-term driving, the physiological data and the behavior indicator of the driver are collected to serve as a basis for subsequent data processing, and can further be classified to improve accuracy of detection.

As an example, collecting the physiological data and the behavior data of the driver includes: collecting change data of at least one of a heart rate, an electroencephalographic signal, and an electromyographic signal of the driver within a predetermined time period as the physiological data; and collecting change data of at least one of a head posture, a blink frequency, an eye opening or closing state, and a facial expression of the driver within a predetermined time period as the behavior data.

It should be understood by those skilled in the art that, the physiological data herein refers to a physiological signal that can be used to measure a person's mental state, including but not limited to the electroencephalographic signal, the electromyographic signal, an electrodermal signal, an electrocardiogramal, a respiratory rate, blood pressure, etc. The behavior data is a record of human body's behavior and data generated when the behavior occurs, and herein refers to a behavior of the driver during driving and recorded data of the behavior. The predetermined time period is a pre-set duration, for example, ten minutes.

The fatigue state of the driver may be effectively identified through various physiological and behavior indicators, such as a brain wave, eye movement, head posture, and heart rate variability. During long-term driving, the physiological and behavior indicators of the driver undergo certain changes, such as a change in an eye movement frequency, a change in the head posture, and a change in the heart rate variability, and these changes are positively correlated with the fatigue state. By collecting and analyzing the various physiological and behavior indicators of the driver, an effective fatigue state identification model can be constructed.

In an embodiment, the heart rate refers to an average number of heartbeats, which varies depending on an age, a gender, a health state, and a living habit. Similarly, the heart rate of the driver differs under different driving conditions. For example, a heart rate under the normal driving state is different from a heart rate under the fatigued driving state. Therefore, the heart rate of the driver within ten minutes during driving may be collected and used as one of the physiological data of the driver.

In an embodiment, the electroencephalographic signal, also known as electroencephalography (EEG), is an electrical signal generated by brain neuron activity. The electroencephalographic signal of the driver during driving also varies with different activities. Subsequent to collecting the electroencephalographic signals of different drivers, these signals are processed using an artificial neural network, to extract and classify typical features of electroencephalography in different frequency bands, determining whether the driver is fatigued. Therefore, the electroencephalographic signal of the driver within ten minutes during driving may be collected and used as one of the physiological data of the driver.

In an embodiment, the electromyographic (EMG) signal is temporal and spatial superposition of motor unit action potentials (MUAPs) among numerous muscle fibers. A surface electromyographic (SEMG) signal is a comprehensive effect of the electromyographic signal from superficial muscle and electrical activity on nerve stem cells on a skin surface, can reflect neuromuscular activity to a certain extent, and is an electrical signal accompanied by muscle contraction. During driving, as the driver makes different body movements, the muscle performs different contraction activities and the electromyographic signal changes accordingly. Therefore, the electromyographic signal of the driver within ten minutes during driving can be collected and used as one of the physiological data of the driver.

In an embodiment, the head posture is generally divided into three types: head raising, head shaking, and head turning. Herein, it can be understood as the head movements of the driver during driving, for example, when a person is drowsy, the head unconsciously droops or tilts to a side. Therefore, the head posture of the driver within ten minutes during driving may be collected and used as one of the physiological data of the driver.

In an embodiment, the blink frequency refers to the number of blinks of the driver within a certain time period during driving. When a person is fatigued, the number of fast blinks, as well as a duration and the number of slow blinks each differ from those in a normal state. Therefore, the blink frequency of the driver during driving, such as the number of blinks of the driver within ten minutes, can be collected and used as one of the physiological data of the driver.

In an embodiment, the eye opening and closing state refers to a state of eyes of the driver during driving, and is used to determine whether the driver is fatigued. For example, a closing duration of the detected eyes of the driver within a unit time period (usually 1 minute or 30 seconds) may be calculated. If the eyes are closed for approximately 80% of the unit time period, it may be determined that the driver is in the fatigue state at this moment. In an embodiment, an aspect ratio of the eyes may be computed by calculating actual pixel values occupied by the detected eyes of the driver in horizontal and vertical directions. This ratio is relatively constant for the eye opening or closing state of the same person, but there is a common feature of this value across different individuals: the ratio is relatively small when the eyes are closed (less than 0.3). For example, if the driver remains in a state where the aspect ratio of the eyes is less than 0.3 for 30 consecutive seconds within one minute, the driver is in the fatigue state at this moment. Therefore, the eye opening and closing state of the driver within one minute during driving may be collected and used as one of the physiological data of the driver.

In an embodiment, the facial expression is an expression of the driver during driving that differs significantly from that when the driver is focused in the normal driving state. For example, if the driver is yawning, in a daze, or the eyes are shedding physiological tears due to fatigue, the driver is in the fatigue state at this moment. Therefore, the facial expression of the driver within ten minutes during driving may be collected and used as one of the physiological data of the driver.

In the embodiments of the present disclosure, different physiological data and behavior data of the driver within a predetermined time period can be collected. Sufficient physiological data and behavior data serve as data support for identifying the fatigue state of the driver in subsequent steps, which effectively ensures the accuracy of the detection, as well as enhances feasibility of the identification and the reliability of the results.

At step S702, at least one independent variable feature of the driver is extracted based on the physiological data and behavior data.

It should be understood that, an independent variable refers to a factor or condition that may be actively manipulated by humans and causes a change in a dependent variable, and may be considered a cause of the dependent variable. For example, if the independent variable is long-term eye closing of the driver, the dependent variable is system's determination that the driver experiences fatigued driving. That is, the cause is the long-term eye closing, and the result is the determination that the driver experiences fatigued driving.

In an embodiment of the present disclosure, the at least one independent variable feature of the driver may be extracted based on the change data of at least one of the collected physiological data, such as the heart rate, the electroencephalographic signal, and the electromyographic signal within a predetermined time period, and the change data of at least one of the collected behavior data, such as the head posture, the blink frequency, the eye opening and closing state, and the facial expression within a predetermined time period.

In the embodiments of the present disclosure, the at least one independent variable feature of the driver is extracted from the physiological data and the behavior data, and subsequent detection is performed based on the at least one independent variable feature, effectively ensuring the accuracy of the detection.

In an embodiment, extracting at least one independent variable feature of the driver based on the physiological data and behavior data includes: preprocessing the physiological data and the behavior data to obtain processed collected data; and extracting at least one energy ratio index from the collected data as the at least one independent variable feature.

In an embodiment, the data preprocessing may include, but is not limited to using EOG artifact removal-independent component analysis (ICA), noise frequency domain filtering-bandpass filtering, and data downsampling-Nyquist sampling theorem.

In an embodiment, feature index screening includes: extracting a feature index that best characterizes the driving fatigue from the preprocessed collected data, where the energy ratio index effectively represent the fatigue state of the driver; extracting an energy feature by frequency decomposition of rhythmic waves using wavelet packet transform, and exploring an index with the strongest correlation with the fatigue state by using grey relational analysis to screen out an optimal feature for studying the driving fatigue.

In an embodiment, analysis of significant feature difference includes: the effectiveness analysis of driver fatigue feature parameters, which is to test whether these parameters have obvious differences in different fatigue states of the driver. Means of parameters under different fatigue states are calculated, and differences between the means are obtained. Whether the feature parameters are different under different states of the driver is tested by using analysis of variance. According to the analysis of variance theory, the differences of feature parameters between different states come from two aspects:
(1) Parameter error caused by fatigue, that is, a difference caused by a change in the fatigue state of the driver, which is referred to as a between-group error. This difference is expressed by calculating a sum of squared deviations between means of the feature parameters in respective groups and a mean of all samples, and recorded as E_{inter-group}.
(2) Random error, that is, a deviation caused by interference during feature extraction, which is referred to as a within-group difference. This difference is expressed by calculating a sum of squared differences between a mean of feature parameters in each group and the respective feature parameters in the group, and recorded as E_{intra-group}.

Mean square values may be obtained by dividing E_{inter-group} and E_{intra-group} by their respective degrees of freedom, which are: a inter-group mean square (MS_{inter-group}) and a intra-group mean square (MS_{intra-group}), respectively. When these two parameters satisfy the first equation below, different fatigue states do not affect the parameter values, that is, samples come from the same population. When the different fatigue states have a relatively significant impact on the extracted feature parameter values, MS_{inter-group} is caused by both the fatigue state of the driver and the random error, that is, the samples come from different populations. In this case, a relationship between MS_{inter-group} and MS_{intra-group} satisfies the equation (3) below.

$$\frac{M {S}_{inter - group}}{M {S}_{intra - group}} \approx 1 ; \qquad ( 2 )$$

$$M {S}_{inter - group} >> M {S}_{intra - group} . \qquad ( 3 )$$

In this case, a ratio of MS_{inter-group} to MS_{intra-group} follows the F-distribution. By comparing an F value with its critical value, whether the samples come from the same population can be determined. Assuming there are n driver fatigue samples, the null hypothesis is that the means under all the fatigue states are the same, that is:

$${u}_{1} = {u}_{2} = {u}_{3} = u ; \qquad ( 4 )$$

where μᵢ represents a mean of the i-th fatigue state. The null hypothesis indicates that n samples come from the same population (i.e., μ and σ are the same). If the calculated result matches a situation shown in equation (3), that is, MS_{intra-group} is much smaller than MS_{inter-group}, it means that the different fatigue states of the driver cause differences between the feature parameter and the mean, that is:

$$F > {F}_{0.05} ( {f}_{inter - group} , {f}_{intra - group} ) ; \qquad ( 5 )$$

where, f in parentheses represents the degree of freedom. Generally, p<0.05 indicates a statistical difference, p<0.01 indicates a statistically significant difference, and p<0.001 indicates a statistically extremely significant difference.

Valid data is collected for use.

In the embodiments of the present disclosure, prior to extracting at least one independent variable feature from the physiological data and behavior data, the physiological data and the behavior data can be pre-preprocessed to ensure validity of the detected data and filter out invalid or redundant data. At least one energy ratio index is extracted from the obtained valid data as the at least one independent variable feature, effectively improving efficiency of data detection and ensuring accuracy and validity of data.

At step S703, an actual fatigue state of the driver is identified based on the at least one independent variable feature, and the vehicle is controlled to issue a state reminder to the driver based on the actual fatigue state.

In an embodiment, subsequent to obtaining the at least one independent variable feature from the above-described embodiments, the actual fatigue state of the driver can be identified. For example, collected and pre-processed heart rate change data within a predetermined time period is used as an independent variable feature. The heart rate of the driver decreases subsequent to driving for a long duration. In this case, it can be determined that the driver is in the fatigue state.

In an embodiment, subsequent to identifying that the driver is in the fatigue state, the fatigue state of the driver may be further classified into levels, which are designated as the actual fatigue state, and the reminder is sent to the driver based on the actual fatigue state of the driver. For example, a corresponding state voice alarm is issued through a voice alarm system, or a reminder is issued through an icon or text on an in-vehicle display screen or through adjustment to interior lighting.

In the embodiments of the present disclosure, the actual fatigue state of the driver can be identified based on the at least one independent variable feature, and the vehicle can be controlled to issue the state reminder to the driver based on the identification result, which ensures the accuracy of the identification result and provides the relevant reminder to prevent driver from fatigued driving, meeting usage needs of the driver in actual scenarios.

In an embodiment, the identifying the actual fatigue state of the driver based on the at least one independent variable feature includes: inputting the at least one independent variable feature into a pre-constructed fatigue state identification model, to obtain the actual fatigue state. The fatigue state identification model is jointly constructed by KSS scores prior to and subsequent to driving, and a plurality of fatigue states.

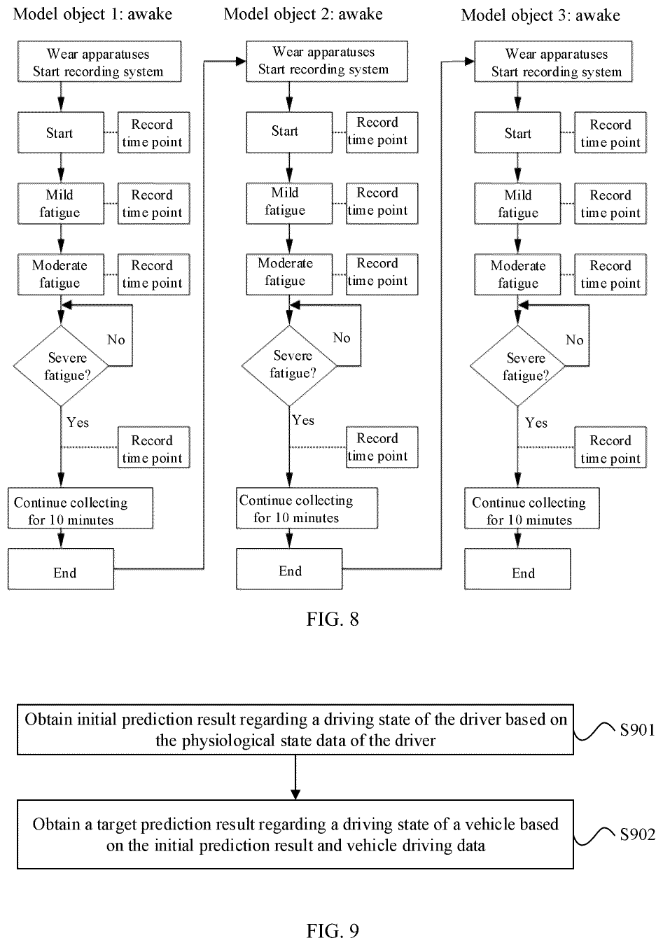

It should be understood based on other relevant embodiments that the fatigue state identification model herein is a pre-trained and established model, and may be, but is not limited to, constructed by the KSS scores prior to and subsequent to driving, and the plurality of fatigue states. As illustrated in FIG. 8, which is a schematic diagram of driver fatigue state identification and monitoring according to an embodiment of the present disclosure. The fatigue state identification model may be constructed as follows.

In an embodiment, model variable control is performed, where independent variables include: a change in the head posture, a change in the blink rate, a change in the eye opening or closing state, a change in the facial expression, a change in the heart rate, a change in the electroencephalographic signal, and a change in the electromyographic signal; and a dependent variable includes the fatigue state (non-fatigue, mild fatigue, moderate fatigue, and severe fatigue).

In an embodiment, model subjects and devices are determined, where nine adults with driving experience are selected as the model subjects, which are required to meet the following criteria: aged between 18 and 60 years old, in good health, with no medical history of psychological, neurological, visual, or other related diseases; and the devices includes: driving simulator system*1, electroencephalography*1, EEG analysis module*1, eye tracker*1, General basic physiological analyzer*1, wireless high-precision physiological recording system*1, camera system*1, head posture detector*1, wearable surface electromyography measurement system*1, EMG analysis module*1, computer system*1, and stopwatch*3;

In an embodiment, model data collection is performed, where subsequent to explaining an operation procedure and precaution to the model subjects, the tester sets test conditions and specifies the types of collected data, and subsequent to adjustment and assembly of the data collection apparatuses, the model subject wears the data collection apparatuses, and continuous collection of the behavior data and the physiological data is started subsequent to ensuring that the physiological signal collection apparatuses are properly connected and turning on the recording system;

Test condition settings include: simulated driving environments, including long-term monotonous high-speed driving, urban road driving, rural road driving, etc.; a driving duration, ranging from 30 minutes to 240 minutes; a fatigue level, including alert, mild fatigue, moderate fatigue, and severe fatigue; and a feature of the model subject, including the age, the gender, driving experience, seat angle adjustment, etc., Types of collected data include behavior data, including a change in the head posture, a change in the blink rate, a change in the eye opening and closing state, and a change in the facial expression; and physiological data, including a change in the heart rate, a change in the electroencephalographic signal, and a change in the electromyographic signal.

In an embodiment, a fatigue state labeling method is performed, including: recording the fatigue state of the model subject during driving and an occurrence duration by using a combination of subjective assessment of the model subject via Karolinska Sleepiness Scale (KSS) and a state recording button, to determine different fatigue state labels corresponding to the collected data samples.

Scores ranging from 1 to 5 are defined to indicate that the driver is in a normal state, while scores ranging from 6 to 10 are defined to indicate that the driver is in the fatigue state, where 6 and 7 indicate mild fatigue, 8 indicates moderate fatigue, and 9 and 10 indicate severe fatigue. The model subjects determine changes in their own states using the KSS. When the states are the mild fatigue, the moderate fatigue, and the severe fatigue, respectively, they signal a recorder, who records time points to facilitate segmentation of the electroencephalographic signal, the electrocardiogramal, and so on. When the model subject reaches the severe fatigue, data collection continues for additional 10 minutes. The collected data is stored and exported, completing the data collection.

It should be noted that, a subjective scale is used as an auxiliary tool, and a state recording and labeling method is mainly adopted. The model subject needs to conduct a subjective evaluation using the KSS scale prior to and subsequent to simulated driving, respectively. By recording time points of state changes of the model subject and combining them with states displayed on the scale prior to and subsequent to the driving, time periods corresponding to four different states of the model subject are determined, to label the collected data. In this way, the fatigue state identification model is obtained.

The fatigue levels are classified into 10 grades in the KSS, where a higher grade represents a deeper degree of fatigue. Table 1 shows the KSS scores and the plurality of fatigue states, as follows:

TABLE 1

| | Fatigue | State |
|---|---|---|
| Mental state | level | classification |
| Extremely alert | 1 | Non-fatigue state |
| Very alert | 2 | |
| Alert | 3 | |
| Somewhat alert | 4 | |
| Neither alert nor drowsy | 5 | |
| Having some signs of drowsiness | 6 | Mild fatigue |
| Drowsy, but still able to stay awake | 7 | |
| Drowsy, requiring effort to stay awake | 8 | Moderate fatigue |
| Very drowsy, requiring considerable | 9 | Severe fatigue |
| effort to stay awake and struggling | | |
| to resist sleep | | |
| Extremely drowsy, unable to stay awake | 10 | |

where, 1 represents extreme alert, 2 represents very alert, 3 represents alert, 4 represents somewhat alert, 5 represents neither alert nor drowsy, 6 represents having some signs of drowsiness, 7 represents drowsy but still able to stay awake, 8 represents drowsy and requiring effort to stay awake, 9 represents very drowsy and requiring considerable effort to stay awake and struggling to resist sleep, and 10 represents extreme drowsy and unable to stay awake. Further, these scores are classified into states as follows: 1 to 5 represent non-fatigue state, 6 and 7 represent mild fatigue, 8 represents moderate fatigue, and 9 and 10 represent severe fatigue.

In the embodiments of the present disclosure, the at least one independent variable feature may be inputted into the fatigue state identification model constructed through pre-training, to obtain the actual fatigue state. The fatigue state identification model is pre-constructed by professional KSS scores and the plurality of fatigue states, and different fatigue levels are classified. Therefore, convergence and reliability of the model are guaranteed through offline training, improving feasibility and reliability of the present disclosure.

In an embodiment, said identifying the actual fatigue state of the driver based on the at least one independent variable feature includes: obtaining a current operating condition and/or current environment of the vehicle; generating a weight for each of the at least one independent variable feature based on the current operating condition and/or current environment; and determining the actual fatigue state based on the at least one independent variable feature and the corresponding weight.

It should be understood that the current operating condition of the vehicle refers to a condition in which the vehicle is traveling on a current road, including, for example, traveling monotonously on a highway, traveling on an urban road, or travelling on a rural road, etc.

In an embodiment, the current operating condition and the current environment of the vehicle both exert an impact on the driving state of the driver. For example, when the vehicle is in a relatively good operating condition, such as traveling monotonously on a highway, the driver is in a relaxed state. In this case, a frequency of the electromyography shows a downward trend, but this does not indicate occurrence of fatigue or an increase in the fatigue level. For example, when the vehicle is exposed to strong light, the eyes of the driver unconsciously squint for a long duration due to glare. In this case, a degree of eye closing is increased, but this does not indicate drowsiness, meaning that it is not the moderate fatigue.

In an embodiment, since different operating conditions and different environments affect the physiological data and the behavior data of the driver, a weight for each of the at least one independent variable feature may be generated based on the current operating condition and/or the current environment. For example, when the current operating condition is relatively good, it is necessary to reduce the weight of the electromyographic signal, while under the strong light, it is necessary to reduce the weight of the eye opening and closing state. The actual fatigue state is further determined based on the at least one independent variable feature and the corresponding weight.

In the embodiments of the present disclosure, based on collection of the physiological data and the behavioral data of the driver and in further combination with the current operating condition and/or the current environment of the vehicle, the actual fatigue state of the driver can be determined, which effectively improves the accuracy of the detection and meets different needs of the driver during actual use.

In an embodiment, the issuing the state reminder to the driver based on the actual fatigue state includes: determining whether the actual fatigue state meets a predetermined reminder condition; and determining a target reminder type and a reminder signal matching the actual fatigue state, and controlling the vehicle to issue the state reminder based on the reminder signal according to the target reminder type, in response to the actual fatigue state meeting the predetermined reminder condition.

It should be understood that the predetermined reminder condition may be a condition that is pre-set and may be used to control the vehicle to issue the reminder to the driver. For example, the predetermined reminder condition may be the driver being in moderate fatigue during driving.

In an embodiment, if the predetermined reminder condition is that the fatigue level of the driver is moderate fatigue or above, when an actual fatigue state of the driver reaches the moderate fatigue, it may be determined that the predetermined reminder condition is met. In this case, the target reminder type and the reminder signal that match the actual fatigue state of the driver may be determined, and the vehicle may be controlled to issue the state reminder based on the reminder signal.

For example, if the actual fatigue state of the driver is mild fatigue, the matched target reminder type is a voice alarm type, and the reminder signal is a broadcast reminder. That is, the vehicle may be controlled to broadcast a voice message through an in-vehicle speaker: “Hello, driver, we have detected that you have been driving continuously for more than 3 hours. Continuing to drive may cause fatigue driving. Please take a break!” For example, if the driver is in the moderate fatigue state, the matched target reminder type is an action type, and the reminder signal is a seat adjustment. That is, a comfortable seat angle that the driver usually sets may be adjusted, such as slightly raising the backrest by 15 degrees, to remind the driver.

In the embodiments of the present disclosure, a predetermined reminder condition may be pre-set, in such a manner that when the actual fatigue state meets the predetermined reminder condition, the state reminder is issued to the driver through the generated reminder type and reminder signal, which reminds the driver at a critical moment to reduce the likelihood of occurrence of safety accidents, improving the driver experience, and meeting the usage needs of the driver in actual scenarios.

In an embodiment, the predetermined reminder condition is that a fatigue level of the actual fatigue state is greater than a predetermined level.

It should be understood that, the predetermined level may be a fatigue state level that is preset and requires the reminder.

For example, if the predetermined level is the mild fatigue, when the driver is in the fatigue state and the fatigue level is the moderate fatigue or severe fatigue, which is higher than the predetermined level, that is, the reminder condition has been met. Therefore, a predetermined state reminder may be issued to the driver.

In the embodiments of the present disclosure, a predetermined reminder condition and fatigue levels may be pre-set, to remind the driver when it is determined that the predetermined reminder condition is met when the actual fatigue level is greater than the predetermined level. Therefore, driving safety is guaranteed, which improves the driver experience, and maintains customer stickiness.

Studies have shown that distraction significantly reduces the ability of the driver to perceive danger, that is, the ability of the driver to respond to danger is reduced. Therefore, it causes the driver to judge the road environment inadequately and inaccurately, resulting in improper driving behavior and ultimately leading to a driving accident. If the driver can be promoted when distracted during driving, some accidents can be effectively avoided. Statistics show that if reaction time of the driver can be advanced by 0.5 seconds, likelihood of the occurrence of the accident is reduced by approximately 60%, and even if the accident does occur, its severity can be reduced.

In an embodiment, the driver state may be predicted using traditional machine learning and deep learning methods. The traditional machine learning method has a small amount of computation and low requirements for a hardware platform, making it suitable for an embedded platform. However, this method requires a complex feature extraction process. The deep learning method can automatically extract an effective feature, which simplifies a classification process, and improves generalization. Also, this method exhibits excellent classification performance with sufficient data. However, it has a large amount of computation and has high requirements for the hardware platform.

Deep learning is an end-to-end algorithm, and is a type of representation learning. It only requires data input and produces a corresponding target output, eliminating complex intermediate feature engineering, and allowing self-learning and extraction of useful features. Due to its advantages, such as the self-learning feature and excellent performance, the deep learning method can be applied to address the problem of driving distraction.

For example, the driver distraction may be detected based on a spatiotemporal convolutional neural network of the electroencephalographic signal. By combining a time domain feature and a frequency domain feature of the electroencephalographic signal, high accuracy is achieved. In addition, compared with a traditional algorithm, this framework demonstrates superior computational efficiency and parameter update speed, making it applicable to an online distraction monitoring system. For example, the fatigue state of the driver may be predicted by constructing a long short-term memory (LSTM) model for detecting the fatigue state of the driver. The LSTM model can make full use of a temporal characteristic of the electroencephalographic signal and can be optimized by combining training process visualization. Compared with a traditional machine learning algorithm, its accuracy is significantly improved. For example, the fatigue state of the driver may be predicted based on a convolutional neural network of channels. This method fully leverages a channel correlation characteristic of the electroencephalographic signal and achieves a good effect. For example, different driving scenarios are designed by constructing a simulated driving experiment, an attention state is evaluated through a braking reaction time of the driver, and the electroencephalographic signal is processed by applying a deep convolutional network, realizing detection of braking intention of the driver under different attention states.

In an embodiment, a driving behavior experiment includes the simulated driving experiment and a real-vehicle experiment. For example, in a real-vehicle environment, image data of the driver during driving, including an image of the normal driving state and an image of the distracted state (making a phone call with left/right hands, sending text messages with left/right hands, taking items from a rear seat of the vehicle, and operating an in-vehicle radio), is collected using a camera and is identified by an image technology. For example, distraction research may be conducted using a driving simulator equipped with visual feedback, auditory feedback, and a vehicle sensor. For example, a vehicle turning experiment may be conducted by using the driving simulator, and driving parameters during driving such as a steering wheel angle, a vehicle speed, a brake pedal signal, a lateral acceleration, environmental road information, and a driving duration are collected to explore a steering pattern under low attention, monitoring driver distraction based on steering performance of the vehicle. Based on this, the real-vehicle experiment may be conducted to eliminate an impact of extreme operation conditions on a steering sensor to enhance robustness.

In an example, with continuous development of computer hardware and the increased availability of large-scale driving distraction data, an application of the machine learning algorithm to the study of driving distraction has become possible. For example, the electroencephalographic signal may be analyzed using one-dimensional discrete wavelets, to obtain various wavelet frequency bands, and statistics of wavelet coefficients of the δ-wave, θ-wave, α-wave, and β-wave frequency bands are extracted as features, that are inputted into a neural network. Attention situations of the driver are classified in combination with a fuzzy model. For example, an electroencephalographic power spectrum and a driving scenario may be combined as input features, a support vector machine (SVM) may be applied, and system parameters may be optimized using a particle swarm algorithm, to obtain a driving distraction detection model adapted to different driving scenarios. For example, a frequency domain feature of the electroencephalographic data may be extracted, and feature dimensionality may be reduced based on principal component analysis (PCA). The feature after dimensionality reduction may be inputted into maximum likelihood estimation (MLE) and a k-nearest neighbor (KNN) model, respectively, for classification. For example, the EEG data of the driver in the distraction state may be collected, its power spectrum may be extracted as a feature, and the feature is inputted into the SVM based on a radial basis function (RBF), constructing a driver attention focus tracking system.

It should be understood that, the driving state of the driver is monitored and predicted through a driver distraction monitoring system, in such a manner that correction is made to the driver prior to occurrence of danger due to the driver distraction, reducing a degree of the driver's erroneous perception and determination of the environment, and shortening the reaction time to dangerous scenarios. These have significant theoretical and practical value for reducing accident rates and improving traffic safety.

In view of the above, the present disclosure provides a driver's driving state prediction method based on multi-modal data. The driver state is predicted by combining the multi-modal data, including physiological state data and vehicle driving data of the driver, and monitoring information is fed back to an advanced driver assistance system (ADAS) to alert or remind a distracted driver. When combined with the existing ADAS, this function can reduce distracted behavior of the driver, improving the driving safety.

FIG. 9 is a flowchart of a driver's driving state prediction method based on multi-modal data according to an embodiment of the present disclosure.

As illustrated in FIG. 9, the driver's driving state prediction method based on the multi-modal data includes following steps S901 to S902.

At step S901, an initial prediction result regarding a driving state of a driver is obtained based on physiological state data of the driver.

At step S902, a target prediction result regarding a driving state of a vehicle is obtained based on the initial prediction result and vehicle driving data.

Accidents are often caused by driver distraction during driving. To improve driving safety, it is necessary to predict the driving state of the driver during driving. For example, physiological state information of the driving state may be collected through a sensor. The collected physiological state information of the driver can reflect attention information of the driver to a certain extent. Based on the physiological state information of the driver, the initial prediction result regarding the driving state is obtained, and the initial prediction result regarding the driving state may be either a distracted driving state or a non-distracted driving state. Based on the physiological state information of the driver, obtaining the initial prediction result regarding the driving state of the vehicle can be predicted using a deep learning network or machine learning method. The target prediction result regarding the driving state is obtained based on the initial prediction result of the driver and the vehicle driving data. The target prediction result may include a prediction result of vehicle's dangerous driving. Whether the vehicle is in dangerous driving can be predicted by combining the initial prediction result regarding the driving state with the vehicle driving data, improving accuracy and efficiency of the prediction. Further, when it is predicted that the vehicle is about to engage in the dangerous driving, the distracted driver is warned or reminded, improving driving safety.

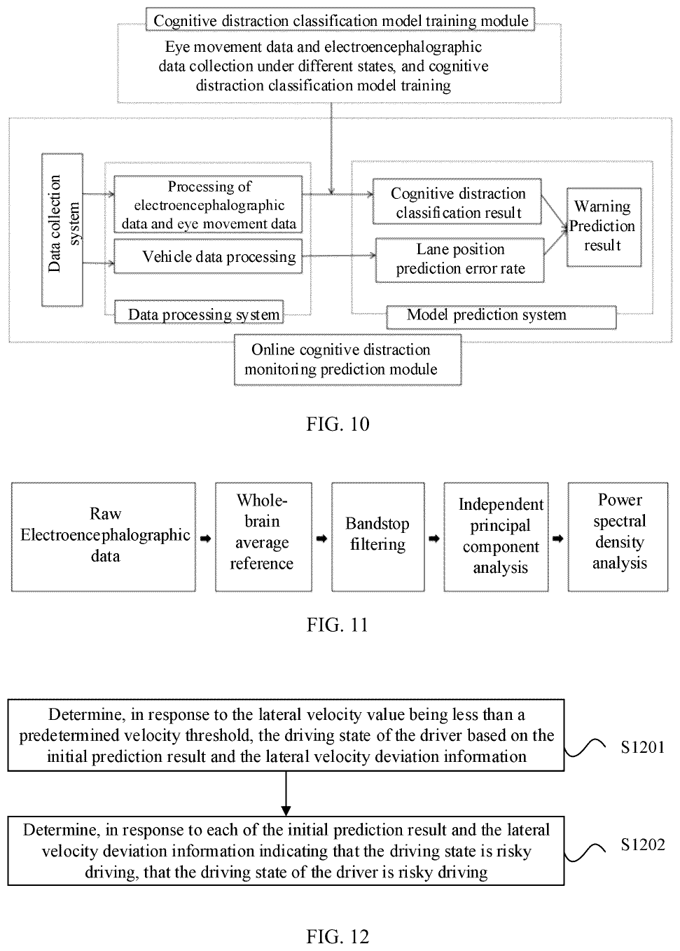

FIG. 10 is a schematic diagram of a driving state prediction system according to an embodiment of the present disclosure.

As illustrated in FIG. 10, the driving state prediction system of the present application includes two modules, where the first module includes a model training module configured to train a deep learning model. The deep learning model may be a cognitive distraction classification model. The first module may be configured to obtain physiological state data under different distracted states as training set data for model training to obtain a cognitive distraction classification model. In an example, the physiological state data includes at least one of eye movement data and electroencephalographic data. In other embodiments of the present disclosure, the physiological state data may also include other types of data, such as movement data of a body part (a hand, a foot, etc.). The movement data of the body part can, to a certain extent, reflect whether the driver is distracted. For example, if the driver uses one hand to operate a mobile phone during driving, the movement data of the hand indicates the driver distraction.

The second module includes an online cognitive distraction detection module, which consists of at least three systems: a data collection system, a data processing system, and a model prediction system. The data collection system is configured to collect the electroencephalographic data, the eye movement data, and the vehicle data (i.e., the vehicle travelling data) in real time. The data processing system is configured to process the data collected in real time, including processing the electroencephalographic data, the eye movement data, and the vehicle travelling data. The processing results regarding the electroencephalographic data and the eye movement data are sent to the cognitive distraction classification model trained by the first module for prediction, obtaining the cognitive distraction classification result (i.e., the initial prediction result). The collected vehicle driving data is processed to obtain a vehicle position prediction error rate. In combination with the prediction error rate, the driving state is predicted using a predetermined rule in the model prediction system.

When performing the prediction based on the electroencephalographic data and the eye movement data, data processing including preprocessing may be performed on the electroencephalographic data and the eye movement data.

In an embodiment, obtaining the initial prediction result regarding the driving state based on the physiological state data of the driver includes: inputting at least one of the eye movement data and the electroencephalographic data into a trained deep learning model for prediction, to obtain the initial prediction result.

The physiological state information of the driver is collected. The physiological state information is used to determine whether the driver is distracted. Therefore, the collected physiological state information includes at least one of the eye movement data and the electroencephalographic data. The at least one of the eye movement data and the electroencephalographic data is inputted into the trained deep learning model, and the deep learning model perform the feature extraction on the inputted eye movement data and electroencephalographic data to predict the driving state, obtaining the initial prediction result. The initial prediction result indicates whether the driver is distracted.

In an embodiment, the trained deep learning model includes the trained cognitive distraction classification model. When this model is trained, a training dataset is obtained. Eye movement data and electroencephalographic data when one or more drivers perform a dual-task driving experiment as well as eye movement data and electroencephalographic data when one or more drivers perform a single-task driving experiment can be collected, respectively. In an initial stage of establishing each classification model, a large amount of data under different scenarios needs to be accumulated. In the present disclosure, eye movement data and electroencephalographic data information when a driver performs the single-task driving experiment and the dual-task driving experiment, as well as eye movement data and electroencephalographic data information when the plurality of drivers perform the single-task driving experiment and the dual-task driving experiment can be collected, respectively. When collecting data of a single driver, multiple collections can be performed and recorded. Further, data of the multiple collections may be compared to remove data with large errors, and a mean of other data may be calculated for storage. The dual task indicates that the driver performs a distraction task simultaneously when executing a driving task, and the single task indicates that the driver only performs the driving task.

Multiple collections at different time points also need to be performed on the data collection of the plurality of different drivers. For example, data information may be collected within a set time interval or at different time periods. For example, data is collected at 6:00, 10:00, 13:00, 15:00, 19:00, 24:00, and 2:00, and so on. The eye movement data and electroencephalographic data under different states are recorded based on different time points. Because different drivers exhibit different distracted states at different time points, comprehensive consideration is required when collecting the training data of the model, to increase completeness and accuracy of the data. In this way, accuracy of the dataset and prediction accuracy of the driving state can be better improved, improving driving safety.

The prediction result of the trained deep learning model may include a labeling result. Data in the dual-task state is labeled 1, indicating cognitive distraction, and data in the single-task state is labeled 0, indicating no cognitive distraction. Subsequent to training, a trained cognitive distraction classification model is obtained. During online prediction, at least one of the eye movement data and the electroencephalographic data is inputted into the trained cognitive distraction classification model for prediction, to obtain the initial prediction result.

In an embodiment, the initial prediction result may include the labeling result. The data in the dual-task state is labeled 1, indicating cognitive distraction, and data in the single-task state is labeled 0, indicating no cognitive distraction.

In an embodiment, preprocessing the eye movement data includes at least one of the following: removing data of abnormal changes in pupil size, pupil occlusion, or artifact at a pupil edge from the eye movement data; removing data indicating deviation in a gaze line of sight from the eye movement data; removing data where a line of sight is outside a region of interest from the eye movement data; and removing data indicating that a saccade angular velocity is greater than a predetermined angular velocity from the eye movement data.

The collected eye movement data requires preprocessing before being inputted into the trained deep learning model, which can improve accuracy of model prediction. The eye movement data may include raw pupil size time series and gaze position information of the user captured by an eye tracker and an on-site camera. The abnormal changes in the pupil size in the eye movement data include non-positive pupil size values, which are caused by loss of an eye target, eyelid occlusion, or blinking.

As an example, to remove the data where the line of sight is outside the region of interest from the eye movement data, a velocity threshold identification fixation filter (I-VT) may be used to extract a fixation feature and a saccade feature. The threshold may be set to 30 degrees. That is, the saccade angular velocity exceeding the threshold of 30 degrees indicates interference caused by head turning, which needs to be removed to identify fixation and saccade eye movement behaviors.

As an example, when removing the data indicating that the saccade angular velocity is greater than the predetermined angular velocity from the eye movement data, the visual angle area is determined as the region of interest by taking a distance between a road center point in front of the vehicle and the eye as a diameter or radius and taking the eye as a center. That is, a line connecting the eye of the driver as a starting point to the center point of the road ahead is taken as an angle bisector, and an area within a 16° visual angle is determined as the region of interest. Data in a non-region of interest is deleted, and only fixation points in the region of interest are recorded.

In an embodiment, preprocessing the electroencephalographic data includes at least one of the following: averaging the electroencephalographic data of a plurality of channels to obtain a mean, and obtaining a difference between the electroencephalographic data of each of the plurality of channels and the mean; performing filtering on the electroencephalographic data and to obtain data within a predetermined frequency band; removing interference data caused by blinking or body movement in the electroencephalographic data; and performing feature on extraction the electroencephalographic data, and obtaining power spectral density feature data for the predetermined frequency band.

The collected electroencephalographic data also requires preprocessing before being inputted into the trained deep learning model subsequent to preprocessing. The electroencephalographic data may be collected using a head-mounted electroencephalograph.

In an embodiment, as shown in FIG. 11, the preprocessing of the electroencephalographic data includes performing whole-brain average reference to the raw electroencephalographic data. That is, the data collected from a plurality of channels is averaged to obtain a mean, and a difference between data of each of the plurality of channels and the mean may be used as whole-brain average reference data. Then, band-stop filtering is performed, with a filter frequency set to 0.5 Hz to 45 Hz. That is, signals outside this frequency band are removed. An independent component correlation algorithm is used to identify and remove interference data caused by blinking or body movement. Since both the blinking and the body movement interfere with the electroencephalographic signal, these interference data need to be removed. Feature extraction is performed through power spectral density analysis to obtain power spectral density feature data for the predetermined frequency band. The power spectral density features including the theta wave ranging from 3 Hz to 7 Hz, the alpha wave ranging from 8 Hz to 12 Hz, and the beta wave ranging from 13 Hz to 30 Hz may be extracted.

In an embodiment, the initial prediction result indicates whether the driver was distracted during driving, and the target prediction result indicates whether vehicle driving deviation occurs due to distraction of the driver during driving.

At least one of the preprocessed eye movement data and electroencephalographic data is inputted into the trained deep learning model to obtain the initial prediction result. The initial prediction result indicates whether the driver was distracted during driving. For example, if the driver was distracted, a label 1 is outputted, and if the driver was not distracted, a label 0 is outputted. To further improve the accuracy of the prediction, the vehicle driving data may be combined for determination. By processing the vehicle driving data, whether the vehicle has deviated can be determined. For example, by combining the initial prediction result and the vehicle driving data, the target prediction result regarding the driving state is obtained. The target prediction result may indicate whether the vehicle driving deviation occurs due to distraction of the driver during driving.

In an embodiment, the vehicle driving data includes lateral velocity deviation information. The obtaining the target prediction result regarding the driving state of the vehicle based on the initial prediction result and vehicle driving data includes: determining, in response to each of the initial prediction result and the lateral velocity deviation information indicating that the driving state is risky driving, that the driving state of the driver is risky driving.

When driving normally, the lateral velocity deviation of the vehicle is relatively stable or small. For example, when the vehicle is traveling in a straight line, a theoretical value of the lateral velocity deviation is zero. However, in the event of an emergency, the driver instinctively turns the steering wheel. In this case, the lateral velocity deviation of the vehicle becomes relatively large. Therefore, whether the driving state is the risky driving can be determined by analyzing the lateral velocity deviation information of the vehicle. The initial prediction result indicates whether the driver is distracted during driving. If the driver is distracted, the driving state of the driver is the risky driving; otherwise, the driving state of the driver is non-risky driving. The target prediction result regarding the driving state is obtained by combining the initial prediction result with the vehicle driving data. When the initial prediction result is the risky driving and a result of determining the lateral velocity deviation information is also the risky driving, the target prediction result regarding the driving state is the risky driving. This risky driving is caused by the vehicle driving deviation due to distraction of the driver.

In an embodiment, the vehicle driving data includes a lateral velocity value and lateral velocity deviation information. Compared with determining the risky driving condition based on the initial prediction result and the lateral velocity deviation information, the risky driving condition may be determined by further considering the lateral velocity value to improve accuracy. As illustrated in FIG. 12, the obtaining the target prediction result regarding the driving state of the vehicle based on the initial prediction result and vehicle driving data includes following steps S1201 to S1202.

At step S1201, in response to the lateral velocity value being less than or equal to a predetermined velocity threshold, the driving state of the driver is determined based on the initial prediction result and the lateral velocity deviation information.

At step S1202, in response to each of the initial prediction result and the lateral velocity deviation information indicating that the driving state is risky driving, the driving state of the driver is determined to be risky driving.

Driving behaviors such as a U-turn and a lane change also affect the lateral velocity deviation information. Therefore, when the lateral velocity deviation is increased due to a normal U-turn or lane change, it can be determined that there is no driving risk. In an embodiment, when the lateral velocity value is less than or equal to the predetermined velocity threshold, i.e., the lateral velocity value is less than or equal to the lateral velocity value during the U-turn or the lane change, and when each of the initial prediction result and the lateral velocity deviation information indicates that the driving state is the risky driving, the driving state of the driver is determined to be the risky driving.

In an embodiment, the lateral velocity value may be directly measured by a speed detector on the vehicle. The predetermined velocity threshold may be set to 2 m/s. The lateral velocity may be denoted as

$${\hat{x}}_{4 , t \vert t}^{f} ,$$

and the lateral velocity value is an absolute value of the lateral velocity

$${\hat{x}}_{4 , t \vert t}^{f} ,$$

and denoted as

$$\vert {\hat{x}}_{4 , t \vert t}^{f} \vert .$$

When the lateral velocity value is less than or equal to 2 m/s, it indicates that the vehicle is not in a driving condition of the U-turn or lane change. In this case, when each of the initial prediction result and the lateral velocity deviation information indicates that the driving sate is the risky driving, the driving state is determined to be the risky driving.

In an embodiment, when the lateral velocity value is greater than the predetermined velocity threshold and the initial prediction result is the risky driving, the driving state is not the risky driving. For example, when the lateral velocity value is greater than 2 m/s, it is not determined as cognitive distraction.

In an embodiment, as illustrated in FIG. 13, the vehicle driving data includes an actual value of kinematic information, the lateral velocity deviation information includes a first lateral velocity deviation value, and the driver's driving state prediction method based on the multi-modal data further includes the following steps S1301 to S1303.

At step S1301, a predicted value of the kinematic information is obtained based on the actual value of the kinematic information.

At step S1302, a prediction error of the kinematic information is obtained based on the actual value of the kinematic information and the predicted value of the kinematic information.

At step S1303, the first lateral velocity deviation value is obtained based on the prediction error.

The vehicle driving data includes the actual value of the kinematic information, which may be directly obtained through collection. The kinematic information may include information such as a position, velocity, and acceleration of the vehicle. The predicted value of the kinematic information is predicted based on the actual value of the kinematic information. The predicted value of the kinematic information may have a one-to-one correspondence with the actual value of the kinematic information. For example, each of the position, velocity, acceleration, and other information of the vehicle at a next moment is predicted to obtain corresponding predicted values. The prediction error of the kinematic information is obtained (the prediction error includes the vehicle position prediction error rate described above) based on the actual value of the kinematic information and the predicted value of the kinematic information. The obtained predicted value at the next moment is compared with the obtained actual value to obtain the prediction error of the kinematic information. The first lateral velocity deviation value is obtained based on the prediction error.

In an embodiment, all vehicle driving data is required to be collected synchronously, that is, all data have the same starting point. When collecting the actual value of the kinematic information, sampling may be performed at a predetermined frequency in Hertz, for example, a frequency of 10 Hz. Zₜ represents an actual value of the collected vehicle kinematic information at time point t. Each Zₜ represents a vector of q-dimensional vehicle driving data (e.g., position, velocity, acceleration, and other multi-dimensional parameters) measured at time point t. Zₜ₋₁ represents all q-dimensional vehicle kinematic information up to time point t−1. The predicted value of the vehicle kinematic information at the time point t may be predicted through a spatial state model based on Zₜ₋₁ (the actual value of all q-dimensional vehicle kinematic information up to the time point t−1). The predicted value of the vehicle kinematic information at the time point t is denoted as \hat{Z}_{t|t-1}, which represents the q-dimensional predicted result including the position, velocity, acceleration, and the like. The prediction error of the kinematic information is obtained based on the actual value of the kinematic information and the predicted value of the kinematic information. A lane position prediction error may be used as an evaluation metric for the prediction error between the actual value and the predicted value, or an average lane position prediction error may be used as the evaluation metric for the prediction error between the actual value and the predicted value. The average lane position prediction error represents a mean of lane position prediction errors at a plurality of time points within a time window. If the lane position prediction error is small, the driving behavior has not significantly deviated. If the lane position prediction error is large, the driving behavior has significantly deviated, which can lead to dangerous driving. The first lateral velocity deviation value is obtained based on the lane position prediction error.

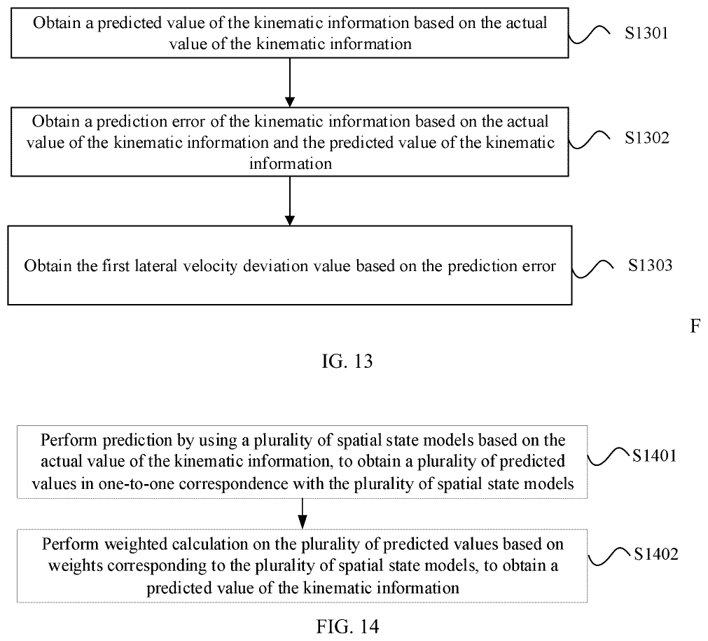

In an embodiment, as illustrated in FIG. 14, obtaining the predicted value of the kinematic information based on the actual value of the kinematic information includes following steps S1401 to S1402.

At step S1401, prediction is performed by using a plurality of spatial state models based on the actual value of the kinematic information, to obtain a plurality of predicted values in one-to-one correspondence with the plurality of spatial state models.

At step S1402, weighted calculation is performed on the plurality of predicted values based on weights corresponding to the plurality of spatial state models, to obtain a predicted value of the kinematic information.

In an embodiment, the actual value of the kinematic information may be predicted using the spatial state model. The spatial state model has a plurality of driving motion modes c∈{1 . . . , C}, where C represents the number of spatial state models, for example, C=6. Six spatial state models include constant velocity (CV), constant acceleration (CA), constant turn rate and velocity (CTRV), constant turn rate and acceleration (CTRA), constant steering angle and velocity (CSAV), and constant curvature and acceleration (CCA). By fusing the plurality of spatial state models, the plurality of predicted values in one-to-one correspondence with the plurality of spatial state models are obtained. In an embodiment, the weighted calculation is performed on the plurality of predicted values to obtain the predicted value of the kinematic information.

In an embodiment, the plurality of spatial state models may be fused using an autonomous multiple model (AMM) algorithm. The fused predicted value is obtained by following equation (6):

$${\hat{z}}_{t \vert t - 1}^{f} = {\sum }_{c = 1}^{C} {w}_{t}^{( c )} {\hat{z}}_{t \vert t - 1}^{( c )} . \qquad ( 6 )$$

Equation (6) indicates that the six spatial state models respectively output six predicted values, and weighted average is performed on the six predicted values to obtain the fused predicted value.

$${w}_{t}^{( c )}$$

represents a normalized weight of each of the six spatial state models at the time point t, and

$$\{ {w}_{t}^{( c )} \vert c = 1 , … , C \} .$$

The weight of each of the six spatial state models may be set as desired. For example, the weights of the spatial state models may be different under different road conditions. The weighted calculation is performed on the predicted values respectively obtained by the six spatial state models to obtain the predicted value of the kinematic information. It should be noted that, the predicted value of the kinematic information is a multi-dimensional predicted value (the q-dimensional including the position, velocity, acceleration, etc.).

The lane position prediction error may be used as the evaluation metric for the prediction error between the actual value and the predicted value, or the average lane position prediction error may be used as the evaluation metric for the prediction error between the actual value and the predicted value.

In an embodiment, the lane position prediction error is denoted as εₜ, which is a difference between the predicted value of the kinematic information, \hat{Z}_{t|t-1} and the actual value of the kinematic information Zₜ, and εₜ is the multi-dimensional lane position prediction error (the q-dimensional including the position, velocity, acceleration, etc.) at the time point t.

In an embodiment, the average lane position prediction error is denoted as ε̅ⱼ, where i represents the i-th time window. One time window includes a plurality of time points. If a time window includes n time points, the average lane position prediction error

$${\overline{ε}}_{i} = \frac{1}{n} {\sum }_{t = n ( i - 1 ) + 1}^{ni} {ε}_{i} .$$

In an embodiment, the first lateral velocity deviation value is obtained based on the lane position prediction error. The first lateral velocity deviation value is calculated using following equation (7):

$${ζ}_{i}^{+} = max \{ 0 , {\overline{ϵ}}_{i} / {σ}_{f} - k + {ζ}_{i - 1}^{+} \} , i = 1 , … , N , \qquad ( 7 )$$

$${ζ}_{0}^{+} = 0 ,$$

$${ζ}_{i}^{-} = max \{ 0 , - {\overline{ϵ}}_{i} / {σ}_{f} - k + {ζ}_{i - 1}^{-} \} , i = 1 , … , N ,$$

$${ζ}_{0}^{-} = 0.$$

where, σ_{ƒ} represents the standard deviation of the lane position prediction error εₜ in a fused state, εₜ in the fused state represents the weighted average of lane position deviation in different states, the weight of each state is the weight corresponding to C spatial state models described above, ε̅ᵢ in formula (2) is obtained by the weighted mean of the lane position deviations in C different states,

$${ζ}_{i}^{+}$$

represents the first lateral velocity deviation value of the i-th time window in a positive direction,

$${ζ}_{0}^{+}$$

represents the first lateral velocity deviation value of the 0-th time window in the positive direction, which is zero,

$${ζ}_{i}^{-}$$

is the first lateral velocity deviation value of the i-th time window in the negative direction,

$${ζ}_{0}^{-}$$

represents the first lateral velocity deviation value of the 0-th time window in a negative direction, which is zero, and k is a predetermined value, which may be set to 0.8.

Prediction is performed on the collected actual value of the kinematic information by using the plurality of spatial state models to obtain the predicted value of the kinematic information, the prediction error of the kinematic information is obtained based on the difference between the actual value of the kinematic information and the predicted value of the kinematic information, and the first lateral velocity deviation value is obtained based on the prediction error. Whether the driving state is the risky driving can be determined through the first lateral velocity deviation value.

In an embodiment, the lateral velocity deviation information indicating that the driving state of the driver is the risky driving includes: determining that the lateral velocity deviation information indicates that the driving state of the driver is the risky driving in response to the first lateral velocity deviation value being greater than the predetermined velocity deviation threshold.

In an embodiment, the first lateral velocity deviation value includes lateral velocity deviation values in two directions: the first lateral velocity deviation value

$${ζ}_{i}^{+}$$

in the positive direction and the first lateral velocity deviation value

$${ζ}_{i}^{-}$$

in the negative direction. The first lateral velocity deviation value being greater than the predetermined velocity deviation threshold includes at least one of the first lateral velocity deviation value

$${ζ}_{i}^{+}$$

in the positive direction and the first lateral velocity deviation value

$${ζ}_{i}^{-}$$

in the negative direction being greater than the predetermined velocity deviation threshold. The predetermined velocity deviation threshold may be set to 8. When at least one of the first lateral velocity deviation value

$${ζ}_{i}^{+}$$

in the positive direction and the first lateral velocity deviation value

$${ζ}_{i}^{-}$$

in the negative direction is greater than 8, it is determined that the lateral velocity deviation information indicates that the driving state is the risky driving.

In addition to obtaining the lateral velocity deviation information through the prediction error, the lateral velocity deviation information may be obtained through the lateral velocity.

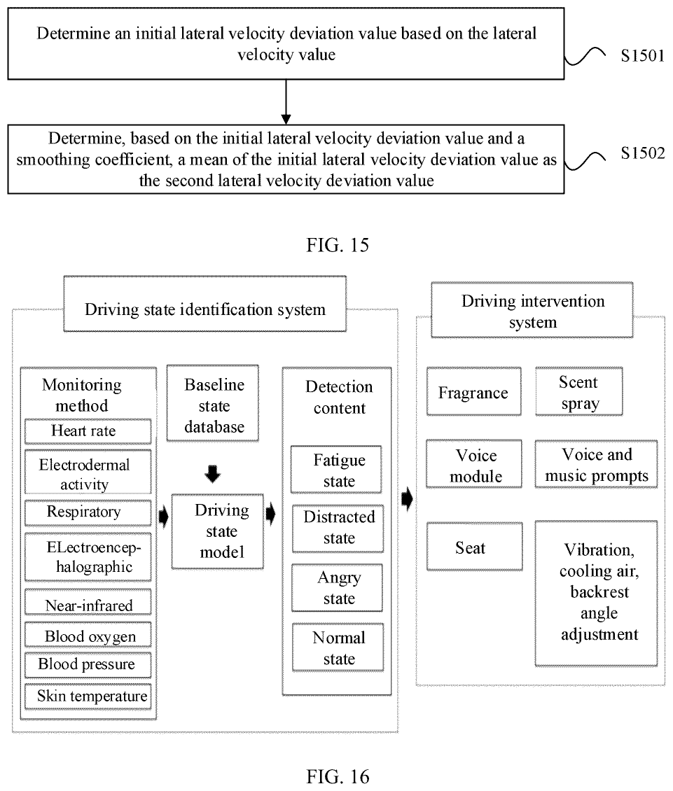

In an embodiment, as illustrated in FIG. 15, the vehicle driving data includes a lateral velocity value, the lateral velocity deviation information includes a second lateral velocity deviation value, and the driver's driving state prediction method based on the multi-modal data further includes following steps S1501 to S1502.

At step S1501, an initial lateral velocity deviation value is determined based on the lateral velocity value.

At step S1502, based on the initial lateral velocity deviation value and a smoothing coefficient, a mean of the initial lateral velocity deviation value is determined as the second lateral velocity deviation value.

In an embodiment, the lateral velocity may be directly measured, and the lateral velocity value is the absolute value of the lateral velocity. The initial lateral velocity deviation value is obtained based on the lateral velocity value, and the initial lateral velocity deviation value is obtained through lateral velocity values within a plurality of windows. For example, a initial time window may be 1 second, and the initial lateral velocity deviation value within the time window i∈{1, . . . . N} is as illustrated in equation (8):

$${ξ}_{i} = \frac{1}{n} {\sum }_{t = n ( i - 1 ) + 1}^{ni} {\hat{x}}_{4 , t \vert t}^{f} . \qquad ( 8 )$$

Next, the mean of the initial lateral velocity deviation value is determined based on the initial lateral velocity deviation value and the smoothing coefficient, as illustrated in equation (9):

$$\overline{ξ} = {λξ}_{i} + ( 1 - λ ) {\overline{ξ}}_{i - 1} , i = 1 , … , N , \qquad ( 9 )$$

$${\overline{ξ}}_{0} = 0.$$

where, ζ̅ represents the mean of the initial lateral velocity deviation value, ζ̅ᵢ represents the initial lateral velocity deviation value in the i-th time window, ζ̅ᵢ₋₁ represents the initial lateral velocity deviation value in the i−1th time window, and λ represents a smoothing parameter. The mean of the initial lateral velocity deviation value may be used as the second lateral velocity deviation value. Whether the driving state is the risky driving is determined based on the second lateral velocity deviation value.

In an embodiment, the lateral velocity deviation information indicating that the driving state of the driver is the risky driving includes: determining that the lateral velocity deviation information indicates that the driving state of the driver is risky driving in response to the second lateral velocity deviation value being within a risk confidence interval.

In an embodiment, the risk confidence interval may be set. If the second lateral velocity deviation value is 0 or relatively small, it indicates a small degree of lateral deviation, that is, there is no risky driving. If the second lateral velocity deviation value falls within the risk confidence interval, it indicates a large degree of lateral deviation, that is, there is risky driving.

In an embodiment, setting the risk confidence interval 1−α may be expressed by formula (10):

$${UCL}_{i} = L {σ}_{0} \sqrt{\frac{λ}{( 2 - λ ) m} [ 1 - {( 1 - λ )}^{2 i} ]} , \qquad ( 10 )$$

$${LCL}_{i} = - L {σ}_{0} \sqrt{\frac{λ}{( 2 - λ ) m} [ 1 - {( 1 - λ )}^{2 i} ]} , i = 1 , … , N ,$$

where, L represents the (1−α/2)-th percentile of the

$${\hat{x}}_{4 , t \vert t}^{f}$$

standard normal distribution, and σ₀ represents the standard deviation; α may be set to 0.0027, and L=3; UCLᵢ represents an upper limit of the risk confidence interval, and LCLᵢ represents a lower limit of the confidence interval. Whether there is risky driving in the driving process is determined by determining whether the second lateral velocity deviation value ζ̅ is within the risk confidence interval. If the second lateral velocity deviation value ζ̅ is outside the risk confidence interval, it indicates that there is no risky driving. If the second lateral velocity deviation value ζ̅ is within the risk confidence interval, it indicates a large degree of lateral deviation, indicating that the risky driving occurs.

By inputting the physiological state data of the driver into the trained cognitive distraction classification model, the initial prediction result is obtained. This initial prediction indicates whether the driver is distracted. The driver being distracted indicates dangerous driving; and the driver being not distracted indicates no dangerous driving. This result is a preliminary prediction result and requires further analysis in combination with the vehicle driving data. The vehicle driving data includes the lateral velocity deviation information. Whether the driving state of the vehicle is the risky driving is determined by calculating the lateral velocity deviation value. The present disclosure provides two exemplary methods for calculating the lateral velocity deviation value, and thus details thereof will be omitted here. By combining the initial prediction result with the vehicle driving data, the target prediction result is obtained. The target prediction result indicates whether the vehicle driving deviation occurs due to the distraction of the driver during driving.

In an embodiment, the determining, in response to each of the initial prediction result and the lateral velocity deviation information indicating that the driving state is risky driving of the driver, that the driving state of the driver is risky driving includes: determining, in response to the initial prediction result indicates that the driving state of the driver is risky driving within a target number of time periods and the lateral velocity deviation information indicates that the driving state of the driver is risky driving, a risky driving level based on the target number. The target number is positively correlated with the risky driving level, and a risk degree corresponding to a higher risk driving level is greater than a risk degree corresponding to a lower risk driving level.

In an embodiment, subsequent to inputting the preprocessed eye movement data and electroencephalographic data in real time, a classification result label is obtained through the cognitive distraction classification model. For example, a classification result label of 1 indicates that the driving state is the risky driving, and a classification label of 0 indicates that the driving state is no risky driving. The lateral velocity deviation information is in units of time windows. For example, the eye movement data and the electroencephalographic data of each time window are inputted into the distraction classification model to obtain the driving state within the time period corresponding to the time window. The risky driving is classified into levels based on the number of time periods (i.e., time windows) in which the driving state is the risky driving. Obviously, the higher level of the risky driving corresponds to the greater number of time periods in which the driving state is the risky driving. That is, the target number is positively correlated with the level of the risky driving, and the risk degree corresponding to the higher level is greater than that corresponding to the lower level.

As an example, the risky driving may be classified into three levels based on the target number, and the target number is denoted as Di. When Di=1, which indicates that a prediction label result corresponding to the i-th time window is 1 (the physiological state data in the i-th time window indicates that the driver is in the distracted state), and the lateral velocity deviation information indicates that the driving state is the risky driving, ADAS issues a Level one risky driving warning. When Di=2, which indicates that each of the prediction label results corresponding to the i-th time window and the (i−1)-th time window is 1 (the physiological state data in the i-th time window and the (i−1)-th time window each indicates that the driver is in the distracted state), and the lateral velocity deviation information indicates that the driving state is the risky driving, the ADAS issues a Level two risky driving warning. When Di=3, which indicates that each of the prediction label results corresponding to the i-th time window, the (i−1)-th time window, and (i−2)-th time window is 1 (the physiological state data in the i-th time window, the (i−1)-th time window, and (i−2)-th time window each indicates that the driver is in the distracted state), and the lateral velocity deviation information indicates that the driving state is the risky driving, the ADAS system issues a Level three risky driving warning. Level three is greater than level two, and level two is greater than level one. The risk becomes greater as the level increases. It should be understood that, more levels of risk levels can be set as desired.

In an embodiment, the driver's driving state prediction method based on the multi-modal data further includes: outputting risk prompting information in a risk warning manner corresponding to the risky driving level.

Different risk prompting manners may be output based on the risky driving level. For example, the outputted prompting information becomes stronger, such as a louder prompting sound, as the risky driving level increases.

It should be noted that, to avoid continuous warnings caused by the same distracted behavior, when the highest level 3 is reached, the first lateral velocity deviation value and the second lateral velocity deviation value are reset to 0.

It should be noted that, when data is unstable or lost, or when sensor data such as the electroencephalographic data or the eye movement data is a null value, Di may be set to 0.

The present disclosure obtains the initial prediction result regarding the driving state based on the physiological state data of the driver; and obtains the target prediction result regarding the driving state based on the initial prediction result and the vehicle driving data. The driver's driving state prediction method based on the multi-modal data can predict the state of the driver by combining the physiological state data of the driver and the vehicle driving data, to warn or remind the distracted driver, improving driving safety.

In an embodiment, a driving mode of the vehicle may be switched based on the target prediction result.

In an embodiment, subsequent to obtaining the target prediction result, the driving mode of the vehicle may be switched based on the target prediction result. For example, the driving mode of the vehicle is initially a conventional driving mode or an assisted driving mode. Under the conventional driving mode, the vehicle is operated and driven by the driver. The assisted driving mode allows the driver to be assisted on the basis of an autonomous driving mode. It can be seen that, switching the driving mode of the vehicle based on the target prediction result can improve the driving safety.

In an embodiment, a driving mode of the vehicle is switched to the autonomous driving mode in response to the target prediction result indicating that the vehicle is in a risky driving state. For example, if the target prediction result indicates that the vehicle is in the risky driving state, it further indicates that when driving in the conventional driving mode or the assisted driving mode, the driver is in a poor driving state, such as being distracted or fatigued. In this case, to ensure the driving safety, switching to the autonomous driving mode is necessary.

In an embodiment, subsequent to switching the driving mode of the vehicle to the autonomous driving mode, when the vehicle is driving in the autonomous driving mode, comfort information of the driver in the autonomous driving mode may be detected in real time. The comfort information indicates whether the driver trusts the autonomous driving mode, whether the driver can relax in the autonomous driving mode, and whether the driver is in a stable mode in the autonomous driving mode.

When the comfort information indicates that a comfort level of the driver is lower than a predetermined level, it indicates that the driver does not sufficiently trust the autonomous driving mode and is in a state of stress, fatigue, or emotional instability in the autonomous driving mode. In this case, the driving mode of the vehicle may be switched from the autonomous driving mode to the assisted driving mode or the conventional driving mode to improve the comfort level of the driver.

In an embodiment, when the vehicle is driving in the autonomous driving mode, at least one of physiological information of the driver and vehicle traveling information may be collected, and the comfort information of the driver may be determined based on the at least one of the physiological information and the vehicle traveling information. The physiological information includes, for example, the electroencephalographic data, emotional data, fatigue data, electrodermal activity data, etc. of the driver. The comfort level of the driver, for example, whether the driver is stressed or fatigued, may be determined by processing the physiological information. The vehicle driving information includes, for example, the steering angle, velocity, acceleration of the vehicle, and whether lane changes occur during driving. The driving information of the vehicle affects the comfort level of the driver to a certain extent, for example, the driver may be in a stressed state when the vehicle makes a sharp turn, travels at a high speed, or changing the lane frequently. Therefore, the physiological information and the vehicle driving information can be combined when detecting the comfort information of the driver.

Another aspect of the present disclosure provides a system for monitoring and feeding back a driving state based on multi-modal human-factor intelligent data analysis, which is also referred to as a driving state monitoring and feedback system based on multi-modal human-factor intelligent data analysis, and a corresponding driving state monitoring and feedback method based on multi-modal human-factor data analysis. The system includes a state identification subsystem and a driving intervention subsystem. The state identification subsystem is configured to receive multi-modal human-factor data collected in real time from a tested driver, preprocess the multi-modal human-factor data; and input the preprocessed multi-modal human-factor data to a pre-trained first state identification model to obtain a driver state identified in real-time. The human-factor data includes at least two of electroencephalographic data, heart rate data, electrodermal activity data, respiratory data, near-infrared data, blood oxygen data, blood pressure data, and skin temperature data. The preprocessing includes noise reduction processing and data normalization processing. The driver state includes a normal state and a plurality of abnormal states. A category of the plurality of abnormal states includes at least two of a fatigue state, a distracted state, and an angry state. The driving intervention subsystem is configured to generate, when the driver state is identified as an abnormal state, a driving state feedback instruction for the category of the abnormal state and send the driving state feedback instruction to a driving intervention system, to cause the driving intervention system to perform state feedback adjustment on the driver based on received driving state feedback instruction.

The first state identification model is obtained by collecting baseline human-factor data and demographic data of the tested driver, retrieving, from a baseline state database, driver-related data having a similarity with the baseline human-factor data and demographic data of the tested driver that is within a predetermined similarity range, and performing iterative training by using retrieved baseline human-factor data of the driver and driver state data stored in the baseline state database as a training set and using a pre-selected normal state and a plurality of pre-selected abnormal states as labels.

In an embodiment of the present disclosure, demographic data of the driver, such as age, gender, height, and weight, is recorded, and baseline human-factor data is collected for five minutes. The baseline human-factor data includes heart rate data, electrodermal activity data, respiratory data, electroencephalographic data, near-infrared data, blood oxygen data, blood pressure data, skin temperature data, etc. During a training process of the first state identification model, based on the demographic data and baseline human-factor data of the driver, similar driver data (the demographic data and baseline human-factor data of the driver, which can be screened according to a predetermined deviation range) is found in a baseline state database preset in the system through a relevant algorithm, and training is performed based on this similar driver data. Classification labels includes a fatigue state, a distracted state, an angry state, and a normal state. During a testing process subsequent to completing the model training, heart rate data, electrodermal activity data, respiratory data, electroencephalographic data, near-infrared data, blood oxygen data, blood pressure data, skin temperature data, and other data are collected and monitored in real time subsequent to correcting a signal through a device for collecting the physiological data of the driver. In a process of identifying the driving state using the first state identification model, the pre-processed data is fed into the trained model to obtain a driving state identification result.

Preprocessing of the electrocardiogramata involves performing noise reduction processing on raw data using a denoising algorithm, such as wavelet filtering, Kalman filtering, and empirical mode decomposition, to remove electromyographic interference, power frequency interference, and perform baseline drift correction, and normalizing the data. Preprocessing of electrodermal activity data involves applying a denoising algorithm to the raw data by taking a value of a time window as a baseline value and a value of another time window as a maximum response value, and normalizing the data. Similarly, noise reduction and data normalization are performed on the respiratory data, the skin temperature data, the blood pressure data, the blood oxygen data, and other data. The data normalization refers to converting data into data that follows a normal distribution. The normal distribution is a common probability distribution characterized by symmetry, uniformity, and predictability. The data normalization can improve the accuracy and efficiency of data analysis and is widely used in fields such as machine learning, statistics, and data mining.

FIG. 16 is a schematic structural diagram of a driving state monitoring and feedback system based on multi-modal human-factor intelligent data analysis according to an embodiment of the present disclosure. FIG. 16 specifically shows components included in a driving state identification system and a driving intervention system.

For the driving state identification system, it monitors the heart rate data, electrodermal activity data, respiratory data, electroencephalographic data, near-infrared data, blood oxygen data, blood pressure data, skin temperature data collected in real time, and inputs these data into the driving state identification system, to obtain the fatigue state, distracted state, angry state, or normal state in the form of labels. The driving state model is obtained through training based on the baseline state database. No processing is required for the label of the normal state. However, for the labels of abnormal states (fatigue, distraction, or anger), in the driving intervention system, a fragrance module is controlled to spray scent through an instruction, a voice module provides voice or music prompts, and the seat is controlled to remind the driver through vibrating, blowing cool air, adjusting a backrest angle, and other manners.

Generally speaking, this system is mounted on a vehicle computer system, which establishes an instruction connection with the fragrance module, voice module, and smart seat in the vehicle.

This system includes two parts: the driving state identification system and the driving intervention system. In the driving state detection system, the physiological data of the driver, including the heart rate data, the electrodermal activity data, the respiratory data, the electroencephalographic data, the near-infrared data, the blood oxygen data, the blood pressure data, and the skin temperature data, can be monitored and collected, and a plurality of states are identified through a model trained by a deep learning algorithm. For example, the plurality of states may include: (1) a fatigue state, (2) a distracted state, (3) an angry state, and (4) a normal state. Subsequent to state identification, early warning and intervention are provided through the fragrance module, the voice module, and the seat in the driving intervention system.

Subsequent to identifying the driving state, the driving intervention system functions according to the identified driving state. When a negative emotional state id detected, feedback can be provided in the form of soothing music or a positive story to adjust the state of the driver accordingly, or the vehicle can be instructed to enter an alert mode and to take emergency action when the driver engages in excessive operations. When the driver is detected to be fatigued, the mood of the driver can be further determined. Since excessive fatigue of the driver, which can lead to a negative emotion, needs to be avoided, further determination is required under this fatigue state, to obtain the physiological information, and further obtain the emotional state. When the emotional state is obtained to be negative, active feedback and adjustment are required. For example, an in-vehicle entertainment system may be activated. The in-vehicle entertainment system may provide music, radio, videos, and other functions to alleviate fatigue and boredom of the driver. These functions can help the driver relax physically and mentally, improving driving comfort and safety. Alternatively, an intelligent voice interaction system may be activated. Through voice recognition and natural language processing technologies of the intelligent voice interaction system, it can interact with the driver and provide services such as navigation, road condition inquiry, and phone call dialing. These services can help the driver better understand traffic conditions and use various vehicle functions, improving driving convenience and safety. Alternatively, synchronized broadcasting may be activated, and vehicle's information may be sent as a marker to other vehicles within a currently set area. In addition, a vehicle-mounted communication system may exchange information with the driver and other vehicles, informing of the vehicle's location and a current driver state. Release of this state is pre-approved by the driver and broadcast within a scope of the approval, preventing occurrence of vehicle accidents. Also, feedback from the vehicle's assisted autonomous driving can be obtained. An intelligent driving assistance system can monitor the state of the driver and vehicle driving conditions, providing early warning and an auxiliary operation such as automatic braking, lane keeping, blind spot monitoring, and adaptive cruise control.

Corresponding to the above-described method, the present disclosure further provides an edge computing terminal device including a computer device. The computer device includes a processor and a memory having computer instructions stored thereon. The processor is configured to execute the computer instructions stored in the memory. The device, when the computer instructions are executed by the processor, implements the above-described method.

In addition, the edge computing terminal device may further include an intelligent sensor and a programmable logic controller (PLC) configured to collect data in the above-described method.

The embodiments of the present disclosure further provide a computer-readable storage medium. The computer-readable storage medium stores a computer program. The computer program, when executed by a processor, implements the above-described method. The computer-readable storage medium may be a tangible storage medium such as a Random Access Memory (RAM), a memory, a Read-Only Memory (ROM), an electrically programmable ROM, an electrically erasable programmable ROM, a register, a floppy disk, a hard disk, a removable storage disk, a CD-ROM, or any other form of storage medium known in the art.

The driving state monitoring and feedback method and system based on multi-modal human-factor intelligent data analysis provided in the present disclosure can process the pre-processed multi-modal human-factor data collected in real time by at least using the pre-trained first-state identification model, distinguish the normal state and the abnormal state of the driver through classification labels, identify different driving states of the driver, and functions according to different driving states to avoid traffic accidents.

What is claimed is:

1. A driving state monitoring and feedback method based on multi-modal human-factor intelligent data analysis, the method comprising:
receiving multi-modal human-factor data collected in real time from a tested driver;
preprocessing the multi-modal human-factor data, wherein the preprocessing comprises noise reduction processing and data normalization processing;
inputting the preprocessed multi-modal human-factor data to a pre-trained first state identification model to obtain a driver state identified in real-time, wherein the driver state comprises a normal state and a plurality of abnormal states, a category of the plurality of abnormal states comprising one or more of a fatigue state, a distracted state, and an angry state; and
generating, in response to the driver state being identified as an abnormal state among the plurality of abnormal states, a driving state feedback instruction for the category of the abnormal state and sending the driving state feedback instruction to a driving intervention system, to cause the driving intervention system to perform state feedback adjustment on the driver based on the received driving state feedback instruction.

2. The method according to claim 1, wherein the multi-modal human-factor data further comprises image data of a head of the tested driver, and the method further comprises, subsequent to preprocessing the multi-modal human-factor data:
analyzing, based on an image identification technology, the image data of the head of the tested driver to obtain an eye movement feature, a head movement feature, and a facial expression feature of the driver; and
analyzing the eye movement feature and the head movement feature of the driver by using an eye tracking technology, and analyzing the facial expression feature by using an expression identification technology, to obtain the driver state identified in real-time.

3. The method according to claim 1, further comprising, subsequent to preprocessing the multi-modal human-factor data:
performing feature extraction on the preprocessed multi-modal human-factor data to obtain a driving behavior-related feature; and
performing label management on the driving behavior-related feature; and performing, when data related to a label is detected, analysis based on the driving behavior-related feature to obtain the driver state identified in real-time;
wherein the driving behavior-related feature comprises one or more of a heart rate, blood pressure, and a respiratory rate.

4. The method according to claim 1, wherein the driver state further comprises a cognitive state of the driver, and the method further comprises, subsequent to preprocessing the multi-modal human-factor data:
obtaining operation data of the driver during driving, the operation data comprising a speed and a force of braking;
obtaining an operation habit of the driver and setting a driver cognitive state identification threshold, based on historical operation data of the driver obtained through historical statistics; and determining that the cognitive state of the driver is abnormal in response to the operation data of the driver collected in real time during driving exceeding the driver cognitive state identification threshold.

5. The method according to claim 1, wherein in response to a virtual driving environment being a multi-task scenario, and the multi-modal human-factor data comprising eye tracking data, the method further comprises:
calibrating a baseline of the collected multi-modal human-factor data;
performing extraction and training on the multi-modal human-factor data during measurement; and
identifying an eye-tracking region of interest based on an eye tracking technology, and extracting a relationship feature between the eye-tracking region of interest and a driving task scenario based on a SEEV model.

6. The method according to claim 1, wherein said generating the driving state feedback instruction for the category of the abnormal state and sending the driving state feedback instruction to the driving intervention system, to cause the driving intervention system to perform the state feedback adjustment on the driver based on the received driving state feedback instruction comprises:
obtaining driver state detection data, vehicle state detection data, and road environment state detection data uploaded by a monitoring terminal, wherein the monitoring terminal comprises a plurality of detection apparatuses, the plurality of detection apparatuses comprising at least a physiological signal detection device, a video signal detection device, and a vehicle signal detection device;
analyzing the driver state detection data, the vehicle state detection data, and the road environment state detection data to obtain a driving state evaluation result; and
performing early warning based on the driving state evaluation result.

7. The method according to claim 6, further comprising:
collecting, when the vehicle travels in a set state along a set route, first state detection data of the driver;
standardizing the collected first state detection data of the driver; and
correlating the driver state detection data obtained in real-time with the standardized first state detection data of the driver.

8. The method according to claim 7, further comprising, subsequent to correlating the driver state detection data obtained in real-time with the standardized first state detection data of the driver:
performing noise processing on the driver state detection data based on the first state detection data of the driver to obtain second state detection data of the driver, wherein the first state detection data of the driver comprises at least a physiological signal, a first eye movement signal, an electroencephalographic signal, a brain imaging signal, and behavior detection data of the driver; and
analyzing the second state detection data of the driver, the vehicle state detection data, and the road environment state detection data to obtain the driving state evaluation result.

9. The method according to claim 8, wherein said performing the noise processing on the driver state detection data based on the first state detection data of the driver comprises:
the driver state detection data comprises a second eye movement signal obtained by parsing video content obtained in real time by the video signal detection device; and
determining artifact and noise in the second eye movement signal in combination with the first eye movement signal, and removing the artifact and noise.

10. The method according to claim 6, wherein said analyzing the driver state detection data, the vehicle state detection data, and the road environment state detection data comprises:
inputting the driver state detection data, the vehicle state detection data, and the road environment state detection data into corresponding state identification models, respectively, to identify the driver state, a vehicle state, and a road environment state; and
combining the driver state, the vehicle state, and the road environment state to obtain the driving state evaluation result.

11. The method according to claim 1, further comprising, subsequent to receiving the multi-modal human-factor data collected in real time from the tested driver:
collecting behavior data of the driver;
extracting at least one independent variable feature of the driver based on the multi-modal human-factor data and the behavior data; and
identifying an actual fatigue state of the driver based on the at least one independent variable feature, and controlling the vehicle to issue a state reminder for the driver based on the actual fatigue state.

12. The method according to claim 11, wherein said extracting at least one independent variable feature of the driver based on the multi-modal human-factor data and the behavior data comprises:
preprocessing the multi-modal human-factor data and the behavior data to obtain processed collected data; and
extracting at least one energy ratio index from the collected data as the at least one independent variable feature.

13. The method according to claim 11, wherein said identifying the actual fatigue state of the driver based on the at least one independent variable feature comprises:
inputting the at least one independent variable feature into a pre-constructed fatigue state identification model, to obtain the actual fatigue state, wherein the fatigue state identification model is jointly constructed by KSS scores prior to and subsequent to driving, and a plurality of fatigue states; or
obtaining a current operating condition and/or current environment of the vehicle; generating a weight for each of the at least one independent variable feature based on the current operating condition and/or the current environment; and determining the actual fatigue state based on the at least one independent variable feature and the corresponding weight.

14. The method according to claim 1, further comprising, subsequent to receiving the multi-modal human-factor data collected in real time from the tested driver:
inputting the multi-modal human-factor data of the driver into a trained deep learning model for prediction, to obtain an initial prediction result regarding a driving state of the driver, wherein the initial prediction result indicates whether the driver is distracted during driving; and
obtaining a target prediction result regarding a driving state of a vehicle based on the initial prediction result and vehicle driving data, wherein the target prediction result indicates whether vehicle driving deviation occurs due to distraction of the driver during driving.

15. The method according to claim 14, wherein the vehicle driving data comprises a lateral velocity value and lateral velocity deviation information, said obtaining the target prediction result regarding the driving state of the vehicle based on the initial prediction result and vehicle driving data comprising:
determining, in response to each of the initial prediction result and the lateral velocity deviation information indicating that the driving state is risky driving, that the driving state of the driver is risky driving; and
determining, in response to the lateral velocity value being less than or equal to a predetermined velocity threshold, the driving state of the driver based on the initial prediction result and the lateral velocity deviation information.

16. The method according to claim 15, wherein the vehicle driving data comprises an actual value of kinematic information, the lateral velocity deviation information comprises a first lateral velocity deviation value, and the method further comprises:
performing prediction by using a plurality of spatial state models based on the actual value of the kinematic information, to obtain a plurality of predicted values in one-to-one correspondence with the plurality of spatial state models,
performing weighted calculation on the plurality of predicted values based on weights corresponding to the plurality of spatial state models, to obtain a predicted value of the kinematic information;
obtaining a prediction error of the kinematic information based on the actual value of the kinematic information and the predicted value of the kinematic information; and
obtaining the first lateral velocity deviation value based on the prediction error, and determining that the lateral velocity deviation information indicates that the driving state of the driver is risky driving in response to the first lateral velocity deviation value being greater than a predetermined velocity deviation threshold.

17. The method according to claim 14, wherein the vehicle driving data further comprises a lateral velocity value, the lateral velocity deviation information comprises a second lateral velocity deviation value, and the method further comprises:
determining an initial lateral velocity deviation value based on the lateral velocity value; and
determining, based on the initial lateral velocity deviation value and a smoothing coefficient, a mean of the initial lateral velocity deviation value as the second lateral velocity deviation value, and determining that the lateral velocity deviation information indicates that the driving state of the driver is risky driving in response to the second lateral velocity deviation value being within a risk confidence interval.

18. The method according to claim 15, wherein said determining, in response to each of the initial prediction result and the lateral velocity deviation information indicating that the driving state is risky driving of the driver, that the driving state of the driver is risky driving comprises:
determining, in response to the initial prediction result indicates that the driving state of the driver is risky driving within a target number of time periods and the lateral velocity deviation information indicates that the driving state of the driver is risky driving, a risky driving level based on the target number,
wherein the target number is positively correlated with the risky driving level, a risk degree corresponding to a higher risky driving level is greater than a risk degree corresponding to a lower risky driving level.

19. The method according to claim 14, wherein said inputting the multi-modal human-factor data of the driver into the trained deep learning model for prediction, to obtain the initial prediction result regarding the driving state of the driver comprises preprocessing eye movement data, said preprocessing eye movement data comprising:
removing data of abnormal changes in pupil size, pupil occlusion, or artifact at a pupil edge from the eye movement data;
removing data indicating deviation in a gaze line of sight from the eye movement data;
removing data where a line of sight is outside a region of interest from the eye movement data; and
removing data indicating that a saccade angular velocity is greater than a predetermined angular velocity from the eye movement data; or
said inputting the multi-modal human-factor data of the driver into the trained deep learning model for prediction, to obtain the initial prediction result regarding the driving state of the driver further comprises preprocessing electroencephalographic data, said preprocessing the electroencephalographic data comprising:
averaging the electroencephalographic data of a plurality of channels to obtain a mean, and obtaining a difference between the electroencephalographic data of each of the plurality of channels and the mean;
performing filtering on the electroencephalographic data to obtain data of a predetermined frequency band;
removing interference data caused by blinking or body movement in the electroencephalographic data; and
performing feature extraction on the electroencephalographic data, and obtaining power spectral density feature data for the predetermined frequency band.

20. An edge computing terminal device, comprising:
a processor; and
a memory having computer instructions stored thereon, wherein the processor is configured to execute the computer instructions stored in the memory, and the device is configured to, when the computer instructions are executed by the processor, implement a driving state monitoring and feedback method based on multi-modal human-factor intelligent data analysis, the method comprising:
receiving multi-modal human-factor data collected in real time from a tested driver;
preprocessing the multi-modal human-factor data, wherein the preprocessing comprises noise reduction processing and data normalization processing;
inputting the preprocessed multi-modal human-factor data to a pre-trained first state identification model to obtain a driver state identified in real-time, wherein the driver state comprises a normal state and a plurality of abnormal states, a category of the plurality of abnormal states comprising one or more of a fatigue state, a distracted state, and an angry state; and
generating, in response to the driver state being identified as an abnormal state among the plurality of abnormal states, a driving state feedback instruction for the category of the abnormal state and sending the driving state feedback instruction to a driving intervention system, to cause the driving intervention system to perform state feedback adjustment on the driver based on the received driving state feedback instruction.
